(19) United States
(12) Patent Application Publication
(10) Pub. No.: US 2006/0199978 A1
(43) Pub. Date: Sep. 7, 2006

(54) Compounds having serotonin 5-ht17 receptor antagonist activity and muscarinic m4 receptor agonist activity and their use in the treatment of psychotic disorders

(76) Inventors: Dimitrios Evangelou Lizos, Glasgow (GB); Clare McKerchar, Glasgow (GB); John Murphy, Glasgow (GB); Yasuyuki Shiigi, Tokyo (JP); Colin Suckling, Glasgow (GB); Hiroshi Yasumatsu, Tokyo (JP); Shen-ze Zhou, Tokyo (JP); Judith Pratt, Glasgow (GB); Brian Morris, Glasgow (GB)

Correspondence Address: WENDEROTH, LIND & PONACK, L.L.P., 2033 K STREET N. W., SUITE 800, WASHINGTON, DC, 20006-1021, US

(21) Appl. No.: 10/551,138

(22) PCT Filed: Mar. 29, 2004
(86) PCT No.: PCT/GB04/01367

§ 371 Date: Mar. 10, 2006

(30) Foreign Application Priority Data
Mar. 29, 2003 (GB) 030737.7
Mar. 29, 2003 (GB) 0307366.5

(51) Int. Cl. A61K 31/155; C07C 251/16

(52) U.S. Cl. 564/245; 514/631

(57) ABSTRACT

The present invention relates to novel treatments for schizophrenia, based on the concept of identifying agents capable of selectively binding to the serotonin 5-HT₇ and muscarinic M₄ receptors and the use of such compounds in treating schizophrenia. The present invention also relates to novel amidine compounds for treating schizophrenia, a method of manufacturing such compounds, pharmaceutical formulations comprising said compounds, as well as medical uses and methods of treatment using said compounds.

### TECHNICAL FIELD

The present invention relates to novel treatments for schizophrenia, based on the concept of identifying agents capable of selectively binding to the serotonin 5-HT₇ and muscarinic M₄ receptors and the use of such compounds in treating schizophrenia. The present invention also relates to novel amidine compounds for treating schizophrenia, a method of manufacturing such compounds, pharmaceutical formulations comprising said compounds, as well as medical uses and methods of treatment using said compounds.

### BACKGROUND ART

The antipsychotic drugs (APDs) currently used in the treatment of schizophrenia are less than optimal in many respects, showing a lack of efficacy against some of the symptoms of schizophrenia and a significant tendency to produce unpleasant side-effects. While all APDs are effective against the positive symptoms of schizophrenia in the majority of patients, they are all less than completely effective against the negative symptoms and cognitive deficits of the disease, with many APDs showing virtually no efficacy against these symptoms. Negative symptoms include loss of emotional responsiveness, lack of motivation and social withdrawal. Cognitive deficits include deficits in working memory, attention and executive function. In addition, in a significant proportion of patients, the positive symptoms which include hallucinations and delusions do not respond to conventional antipsychotic drugs. All current APDs share the common property of affinity and antagonist action at D₂ dopamine receptors (Seeman, 2001). This is thought to underly their activity against the positive symptoms, but unfortunately is responsible also for unpleasant side-effects such as parkinsonian motor deficits and hyperprolactinaemia.

It is widely accepted that clozapine shows the most favourable therapeutic profile of current antipsychotic drugs used in the treatment of schizophrenia. While all APDs, including clozapine, are effective to some degree against the positive symptoms of schizophrenia, clozapine is more effective than other APDs against the negative symptoms and cognitive deficits of the disease, and is also effective in many patients who do not respond to conventional APDs. However, despite its high clinical efficacy, clozapine exhibits relatively low occupancy of D₂ dopamine receptors. In common with most APDs, clozapine binds to many different neurotransmitter receptors implicated in psychosis.

Muscarinic M₄ receptors (Eglen, 2001) are located in brain regions that have been implicated in psychosis, including the prefrontal cortex, and are present in the specific neurones which are compromised in the post-mortem prefrontal cortex tissue from schizophrenic patients. While most APDs either have no affinity for the M₄ receptor or act as antagonists, there is some evidence that M₄ agonists may show APD-like activity in some tests. This is consistent with evidence that the levels of M₄ receptors may be reduced in prefrontal cortex from schizophrenic patients as compared to normal controls (Crook et al., 2001). In addition, serotonin 5-HT₇ receptors (Vanhoenacker et al., 2000) are strikingly localised to thalamic nuclei. Some of the more effective atypical APDs have significant 5-HT₇ affinity as part of their complex pharmacological profile.

There is a need for effective APDs which are able to ameliorate both positive and negative symptoms and the cognitive deficits of schizophrenia and/or bipolar disorder without significant D₂ affinity.

### DISCLOSURE OF INVENTION

Therefore it is a first object of the present invention to obviate and/or mitigate the deficiencies associated with current antipsychotic drug treatments.

It is a second object of the present invention to provide a novel pharmaceutical agent which combines serotonin 5-HT₇ receptor antagonist activity and muscarinic M₄ receptor agonist activity for use in the treatment of schizophrenia and/or bipolar disorder.

It is a third object of the present invention to provide the abovementioned pharmaceutical agents which additionally possess relatively low or negligible dopaminergic D₂ affinity which are useful as antipsychotic agents useful for the treatment of schizophrenia and/or bipolar disorder.

It is a fourth object of the present invention to provide an agent which represents a novel class of antipsychotic drug, useful for the treatment of schizophrenia and/or bipolar disorder.

It is fifth object of the present invention to provide an agent according to the third object which additionally possess relatively low or negligible dopaminergic D₂ affinity which represents a novel class of antipsychotic drug, useful for the treatment of schizophrenia and/or bipolar disorder.

It is a sixth object of the present invention to provide at least one novel amidine compound which possesses serotonin 5-HT₇ receptor antagonist activity and/or muscarinic M₄ receptor agonist activity.

It is a seventh object of the present invention to provide at least one novel amidine compound which additionally possesses relatively low or negligible dopaminergic D₂ affinity.

It is an eighth object of the present invention to provide a pharmaceutical composition comprising said agents for the treatment of schizophrenia and/or bipolar disorder.

A further object of the present invention is to provide a method for identifying an agent as defined above.

The present inventors have hypothesised that the favourable therapeutic profile of clozapine might be based on its 5-HT₇ antagonist activity and muscarinic M₄ agonist activity with low occupancy of D₂ dopamine receptors. According to this hypothesis, an agent possessing 5-HT₇ antagonist activity and substantial muscarinic M₄ agonist activity, yet without significant D₂ dopamine affinity, is postulated to show antipsychotic efficacy against both positive and negative symptoms and cognitive deficits. Such an agent may show an improved therapeutic profile relative to existing APDs, in terms of improved clinical efficacy and reduced side effect profile.

We therefore hypothesised that the combination of these two unusual properties—5-HT₇ antagonist activity and muscarinic M₄ agonist activity—might act to restore disturbed function in the brains of schizophrenic patients. Furthermore, we hypothesised that 5-HT₇ antagonist activity and muscarinic M₄ agonist activity alone, in the absence of D2 antagonist activity, might be sufficient to bestow effective APD activity on such a pharmacological agent. According to this hypothesis, a compound possessing 5-HT₇ antagonist activity and substantial muscarinic M₄ agonist activity, yet without significant D2 dopamine antagonist activity, would show antipsychotic efficacy against both positive and negative symptoms. Such a compound would be predicted to show an improved therapeutic profile relative to existing APDs, in terms of improved clinical efficacy and reduced side effect profile.

Furthermore, schizophrenic patients show marked deficits in cognitive tests, particularly those that are prefrontal cortex dependent, and this is thought to contribute to their inability to lead a relatively normal life. Since there is evidence that M₄ muscarinic agonists should act as cognitive enhancers (Jerusalinsky et al., 1998), a drug with substantial M₄ agonist activity should also be effective against the cognitive impairment characteristic of the disease.

Hence, the present inventors sought to demonstrate that potential therapeutic efficacy from a pharmacological agent combining selectivity versus other receptors with serotonin 5-HT₇ antagonist activity and muscarinic M₄ agonist activity—hereinafter termed a “serominic” compound.

Thus, in a first aspect of the present invention there is provided a pharmaceutical agent having serotonin 5-HT₇ receptor antagonist activity and muscarinic M₄ receptor agonist activity, for use in treating psychotic conditions such as schizophrenia and/or bipolar disorder, wherein the agent does not include compounds having a chemical structure falling within the following definition, namely:

bisarylazepines substituted at the azepine ring portion by a 4-methyl piperazinyl, wherein the aryl moieties are fused to the azepine ring and wherein aryl is phenyl, substituted phenyl, thienyl or substituted thienyl; including optional replacement of an azepine ring carbon atom with a nitrogen atom, or substitution of said ring carbon atom.

The compounds not encompassed by the present invention are represented by the following general formula:

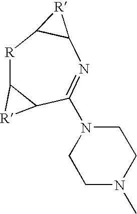

wherein R represents substituted or unsubstituted C or N and each R′ together with the carbon to which it is bonded independently represents phenyl, substituted phenyl, thienyl or substituted thienyl.

The above disclaimer is intended to exclude in particular any accidental anticipation by the compounds clozapine, fluperlipine, tenilapine and olanzapine. These compounds are four known antipsychotic drugs which display M₄ agonism and 5-HT₇ antagonism as part of their wide spectrum of pharmacological actions. Thus, the compounds also show affinity for a large number of receptors, such as adrenergic α₁, α₂; histaminergic H₁, H₂, H₃; dopaminergic D₁, D₂, D₃, D₄, D₅; muscarinic cholinergic M₁, M₂, M₃, M₄, M₅; serotonergic 5-HT_{1A}, 5-HT_{2A}, 5-HT_{2B}, 5-HT_{2C}, 5-HT₃, 5-HT₆, 5-HT₇. As such there is no suggestion in the art that the activities towards the M₄ and 5-HT₇ receptors alone or together are significant or for that matter that the compounds are selective in their action i.e. do not act on many diverse receptors. Moreover, only the four compounds mentioned above out of the large number of atypical antipsychotic drugs show a very weak agonist activity at muscarinic M₄ receptors. While it has been suggested that M₄ agonists or 5-HT₇ antagonists individually may have some therapeutic efficacy against the positive symptoms of schizophrenia, based on results in animal models (Bymaster et al., 1998; Shannon et al., 1999a,b; Pouzet et al., 2002), M₄ agonists or 5-HT₇ antagonists individually have failed to show activity in animal models predictive of efficacy against the negative symptoms of schizophrenia (Bymaster et al., 1998; Pouzet et al., 2002). In view of the very large number of receptors potentially linked to the treatment of schizophrenia, which would include adrenergic α₁, α₂; histaminergic H₁, H₂, H₃; dopaminergic D₁, D₃, D₄, D₅; muscarinic cholinergic M₁, M₂, M₃, M₄, M₅; and serotonergic 5-HT_{1A}, 5-HT_{2A}, 5-HT_{2B}, 5-HT_{2C}, 5-HT₃, 5-HT₆, 5-HT₇ receptors, in addition to D₂ receptors, there is no suggestion in the art that the specific combination of activities just towards the M₄ and 5-HT₇ receptors is significant for the treatment of schizophrenia. Since most clinically useful atypical antipsychotic drugs do not show M₄ agonist activity it is likely that the skilled artisan would not generally believe this property to be important clinically. It has never before been suggested, or demonstrated, that combining the properties of M₄ agonism and 5-HT₇ antagonism, in the absence of any other pharmacological activity, would give activity against all the range of symptoms of schizophrenia.

As used herein the term agonist refers to a ligand that, upon binding to said receptor, triggers activation of a chemical signalling cascade that results in a definable change in the behaviour or physical or biological state of a cell (including partial agonists which cause detectable but sub-maximal activation of signalling cascades) and the term antagonist refers to a molecule that, by virtue of binding to said receptor, is able to block the cell-activating influence of an agonist to said receptor, and which itself does not result in substantial activation of the cell.

The pharmaceutical agent may comprise a mixture of at least two compounds, wherein at least one of said compounds possesses serotonin 5-HT₇ receptor antagonist activity and wherein at least one of said compounds possess muscarinic M₄ receptor agonist activity.

Alternatively, the pharmaceutical agent may comprise a compound which possess both serotonin 5-HT₇ receptor antagonist activity and muscarinic M₄ receptor agonist activity, hereinafter termed a serominic compound.

Preferably the pharmaceutical agent additionally has a low or substantially no dopaminergic D₂ receptor affinity.

A low dopaminergic D₂ receptor affinity may be, for example, a minimum of at least 5 fold less than the affinity at the muscarinic M₄ and/or serotonin 5-HT₇ receptors.

More preferably the dopaminergic D₂ receptor affinity is at least 5 fold, preferably at least 10 or 20 fold or at least 50 fold less than the affinity at the muscarinic M₄ and/or serotonin 5-HT₇ receptors.

In a second aspect of the present invention there is provided a compound represented by formula (I):

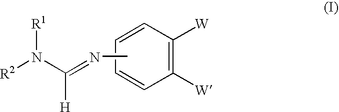

wherein R¹ and R² independently are a hydrogen atom, a substituted or unsubstituted straight chain or branched chain C₁₋₆ alkyl group or C₁₋₆ alkoxy group, a substituted or unsubstituted C₃₋₈ cycloalkyl group or a C₃₋₈ cycloalkoxy group, or an aralkyl group, or R¹ and R² form, together with the nitrogen atom to which they are bonded, a cyclic amine; W and W′ form, together with the benzene ring to which they are bonded, a fused five-membered, six-membered or seven-membered saturated carbocyclic ring being independently unsubstituted, substituted or fully substituted at each carbon atom of the ring by a group —X—R¹³ wherein X is O, S, SO or SO₂ and R¹³ is a hydrogen atom, a C₁₋₆ alkyl group, an acyl group, or an aroyl group or two of said —X—R¹³ groups, together with the carbon atom in the ring to which they are both bonded, form a C═O group, a C═S group or the following group: 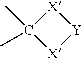
wherein both of X′ are O or S and Y is a C₁₋₃ alkylene group.

The cyclic amine may be substituted by a halogen atom, a C₁₋₆ alkyl group or a C₁₋₆ alkoxy group. Alternatively or additionally, the cyclic amine may be fused with a benzene ring. Said benzene ring may be substituted by one or two halogen atoms, C₁₋₆ alkyl groups or C₁₋₆ alkoxy groups.

The term “substituted” as used herein when in association with the saturated carbocyclic ring refers to one hydrogen atom of a carbon atom of the ring being replaced by a substituent, whereas the term “fully substituted” refers to both of the hydrogen atoms of a carbon atom of the ring being replaced by substituents.

The present inventors hypothesised that exemplary compounds may contain the following features:
a framework that contains an N⁺ or a latent N⁺
a 5-HT₇ responsive group, which would typically be an aromatic system possibly with alkoxy substituents
an M₄ responsive group.

Further compounds of the second aspect of the present invention are represented by the following formulae (II), (III) and (IV) which fall within general formula (I):

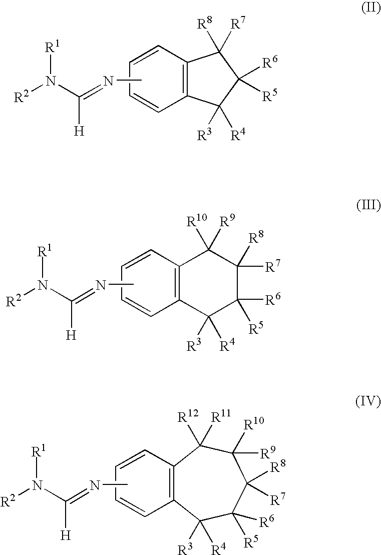

In formulae (II), (III) and (IV), R³, R⁴, R⁵, R⁶, R⁷, R⁸, R⁹, R¹⁰, R¹¹ and R¹² are independently a hydrogen atom or the group —X—R¹³ wherein X is O, S, SO or SO₂ and R¹³ is a hydrogen atom, a C₁₋₆ alkyl group, an acyl group, or an aroyl group.

Alternatively, R³ and R⁴, R⁵ and R⁶, R⁷ and R⁸, R⁹ and R¹⁰, and R¹¹ and R¹² together with the carbon atom in the ring to which they are both bonded, form a C═O group, a C═S group or the following group: 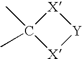
wherein both of X′ are O or S and Y is a C₁₋₃ alkylene group.

At each occurrence in formulae (I), (II), (III) and (IV) examples of C₁₋₆ alkyl groups are methyl, ethyl, propyl, isopropyl, butyl, tert-butyl, pentyl and hexyl.

Examples of aralkyl groups are benzyl, phenylethyl, chlorobenzyl, methylbenzyl, and methoxybenzyl. Examples of halogen atoms are chlorine, bromine, fluorine and iodine.

Examples of C₁₋₆ alkoxy groups are methoxy, ethoxy, propoxy, butoxy, pentyloxy and hexyloxy.

An example of an acyl group is a C₂₋₆ alkanoyl group for example an acetyl, propionyl, butyryl, pentanoyl or hexanoyl.

Examples of aroyl groups are benzoyl, phenylacetyl, chlorobenzoyl, methylbenzoyl, methoxybenzoyl, dichlorobenzoyl, dimethylbenzoyl or dimethoxybenzoyl.

Examples of C₁₋₃ alkylene groups are methylene, ethylene, propylene or trimethylene.

Examples of C₃₋₈ cycloalkyl groups are cyclopropyl, cyclobutyl, cyclopentyl, cyclohexyl, cycloheptyl or cycloctyl optionally substituted by one or more substituents selected from the group consisting of a halogen atom, a C₁₋₆ alkyl group and C₁₋₆ alkoxy group.

Examples of C₃₋₈ cycloalkoxy groups are cyclopropyloxy, cyclobutyloxy, cyclopentyloxy, cyclohexyloxy, cycloheptyloxy or cycloctyloxy optionally substituted by one or more substituents selected from the group consisting of a halogen atom, a C₁₋₆ alkyl group and C₁₋₆ alkoxy group.

Preferred compounds, although not exclusively, are those represented by the above formulae when R¹ and R² form together with the nitrogen atom to which they are bonded, a four-membered, five-membered or six-membered cylic amine.

The six-membered cyclic amine is preferably fused with a benzene ring, typically at carbon atoms 4a and 8a (according to isoquinoline numbering nomenclature).

The said benzene ring may also be substituted at any two adjacent carbon atoms.

Preferably said substitution is with a C₁₋₆ alkoxy group which is preferably a methoxy group.

Alternatively, R¹ and R² may both be a C₁₋₆ alkyl group.

Preferably the alkyl group is a methyl group.

In a further embodiment, R¹ may be an aralkyl group, preferably a benzyl group and R² may be a C₁₋₆ alkyl group, preferably a methyl group.

The five-membered, six-membered or seven-membered saturated (except at the ring fusion) carbocyclic ring is typically substituted by a hydroxyl or an O-acyl group.

Preferably the acyl part of the O-acyl group is a C₂₋₆ alkanoyl group such as an acetyl group or a propionyl group.

Typically the substitution is at carbon number 5 of the seven-membered benzocycloheptyl ring systems and carbon number 1 of the five-membered indanyl and six-membered tetrahydronaphthalenyl ring systems.

Alternatively, the five-membered, six-membered or seven-membered saturated carbocyclic ring may be substituted with an O-aroyl group in which the aroyl part is typically a benzoyl group. The benzene ring of the benzoyl group may be further substituted with halogen atoms such as chlorine atoms. Typically two chlorine atoms are present, preferably at positions 3 and 4 of the benzene ring.

The carbocyclic ring may instead be substituted with a thiol group or a thio group such as a C₁₋₆ alkylthio group. A typical group is a butylthio group.

Alternatively the carbocyclic ring may be substituted by the group —X—R¹³ when it forms the group:

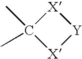

Preferably X¹ is S and Y is a C₂ alkylene group i.e. an ethylene group.

Examples of preferred compounds of the present invention are represented by the following formulae, some of which are named and ring positions numbered to indicate placement of substituents as used herein within the structural formulae.

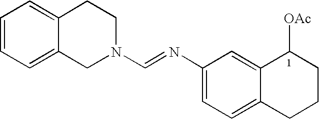

### Acetic acid 7-[(3,4-dihydro-1H-isoquinolin-2-yl methylene)-amino]-1,2,3,4-tetrahydronaphthalen-1-yl ester

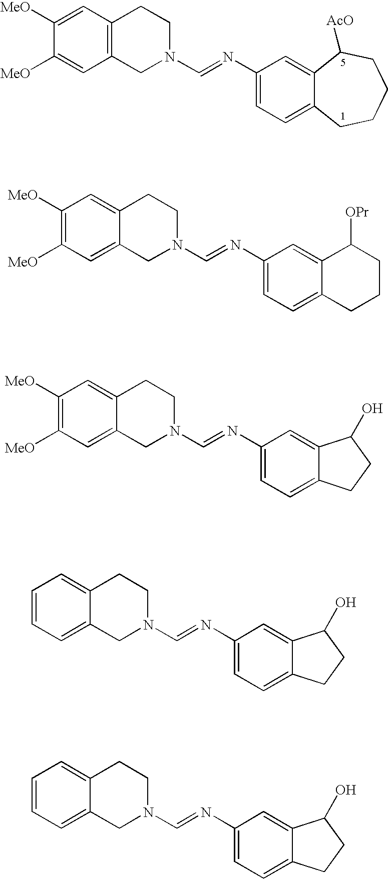

### 6-[(3,4-dihydro-1H-isoquinolin-2-ylmethylene)-amino]-indan-1-ol

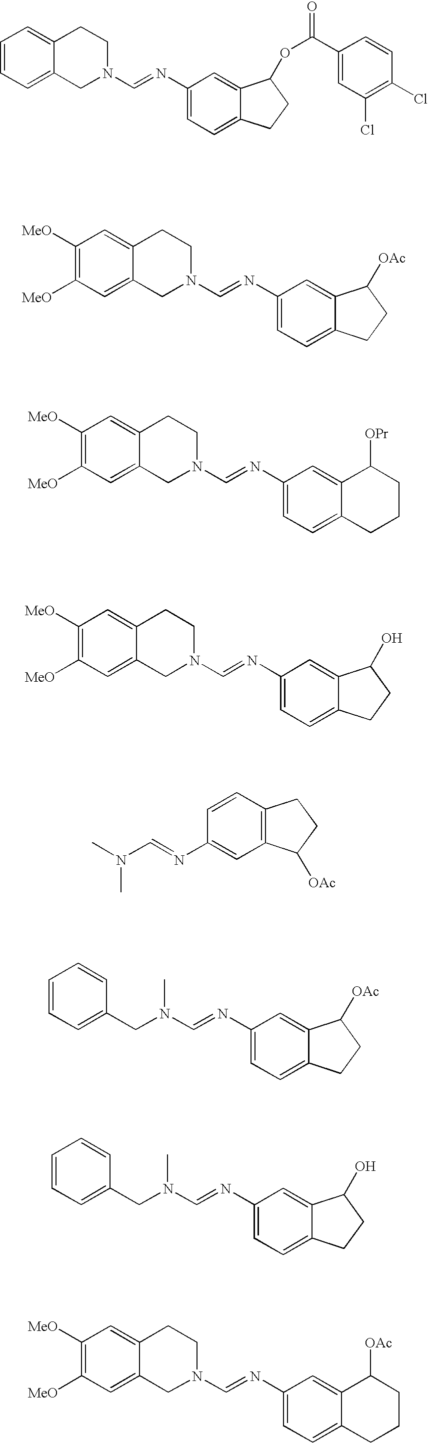

### Acetic acid 6-[(3,4-dihydro-1H-isoquinolin-2-yl methylene)-amino]-indan-1-yl ester

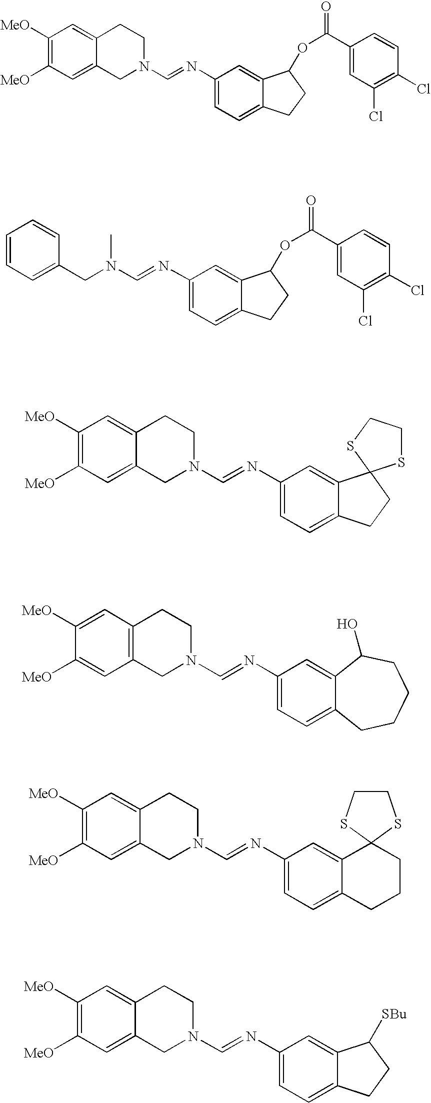

Preferably the said compounds according to any of the formulae (I), (II), (III) or (IV) possess serotonin 5-HT₇ receptor antagonist activity and/or muscarinic M₄ receptor agonist activity.

Preferably the compounds additionally have a low or substantially no dopaminergic D₂ receptor affinity.

A low dopaminergic D₂ receptor affinity may be, for example, a D₂ receptor affinity having a minimum of at least 5 fold less than the affinity for the muscarinic M₄ and/or serotonin 5-HT₇ receptors.

More preferably the dopaminergic D₂ receptor affinity is a D₂ receptor affinity is at least 10 or 20 fold or at least 50 fold less than the affinity for the muscarinic M₄ and/or serotonin 5-HT₇ receptors.

For the avoidance of doubt the compounds of the present invention may be provided as pharmaceutically acceptable salts, solvates inclusive of hydrates or derivatives such as esters.

It is understood that the present invention extends to each of the stereoisomers of the compounds of formulae (I), (II), (III) and (IV) as well as the racemates.

According to a third aspect of the present invention, the amidine compounds represented by formulae (I), (II), (III) and (IV) may be prepared by:

(i) providing an aromatic amine compound;

(ii) providing a formamide compound; and

(iii) coupling the aromatic amine with the formamide to give said amidine compound.

The formamide may be made by condensing an amine with an anhydride derived from formic acid.

The aromatic amine may be produced by reduction of an aromatic nitro compound, which can be prepared by nitration of an arene.

The compounds of formulae (I), (II), (III) and (IV) and their pharmaceutically acceptable salts and/or hydrates can be prepared according to the following procedure for the coupling of amine and formamide, hydrolysis of ester and, if necessary, preparation of salt and/or hydrate form of amidine:

(1) To a solution of formamide (2.0 eq.) in dry dichloromethane (5 mL/mmol of amine) under nitrogen at room temperature was added phosphorus oxychloride (2.0 eq.) dropwise. The solution was stirred at room temperature for 30 min. The resulting solution was transferred to a flask containing amine (1.0 eq) via a cannula under nitrogen and the reaction continued at room temperature for 2 to 3 h. The mixture was diluted with dichloromethane and washed with sodium hydroxide solution (2M), dried over magnesium sulfate, filtered and concentrated. Purification by flash chromatography with suitable eluent afforded the corresponding amidine (base form). Yields ranged from 40 to 60%.

(2) The hydrolysis of some of the coupling product (acetate esters) was performed by dissolving samples in methanol containing a catalytic amount of potassium carbonate at room temperature. The reaction was followed by TLC. The solvent was removed and the residue was dissolved in dichloromethane, washed with water, dried over magnesium sulfate, filtered and concentrated. Purification by flash chromatography gave alcohols.

(3) The salt form of the amidine was made by dissolving the amidine free base sample in dichloromethane and washing with, for example hydrochloric acid (2M), and drying over magnesium sulfate. Filtration and concentration afforded the corresponding salt form of amidine.

Emerging evidence suggests that schizophrenia results from dysfunction of specific neural circuits in the brain. There is pathological evidence for dysfunction of cells in the prefrontal cortex in schizophrenic patients, along with clear indications from brain imaging studies that the prefrontal cortex is hypofunctional. The prefrontal cortex is a key part of the corticolimbothalamic circuit, which connects it both directly and indirectly to the midline thalamic nuclei. Dysfunction of this circuit in schizophrenia is consistent with the concept that the symptoms of schizophrenia are due to perturbation of midline thalamic function. Without wishing to be bound by theory, the present inventors therefore hypothesised that a pharmacological agent able to restore disturbed thalamic and prefrontal cortex function may effectively treat the symptoms of schizophrenia.

Muscarinic M₄ receptors (Eglen, 2001) are located in brain regions that have been implicated in psychosis, including the prefrontal cortex, and are present in the specific neurones which are compromised in the post-mortem prefrontal cortex tissue from schizophrenic patients. While most APDs either have no affinity for the M₄ receptor or act as antagonists, there is some evidence that M₄ agonists may show APD-like activity in some tests. This is consistent with evidence that the levels of M₄ receptors may be reduced in prefrontal cortex from schizophrenic patients as compared to normal controls (Crook et al., 2001). In addition, serotonin 5-HT₇ receptors (Vanhoenacker et al., 2000) are strikingly localised to thalamic nuclei. Some of the more effective atypical APDs have significant 5-HT₇ affinity as part of their complex pharmacological profile.

The present inventors therefore considered it possible that the combination of the two unusual properties—5-HT₇ antagonist activity and muscarinic M₄ agonist activity—might act to restore disturbed function in the brains of schizophrenic patients. Furthermore, they hypothesise that 5-HT₇ antagonist activity and muscarinic M₄ agonist activity, in the absence of D₂ affinity might be sufficient to bestow effective APD activity on such a pharmacological agent. According to this hypothesis, an agent possessing 5-HT₇ antagonist activity and substantial muscarinic M₄ agonist activity, yet without significant D₂ dopamine affinity, is postulated to show antipsychotic efficacy against both positive and negative symptoms and cognitive deficits. Such an agent may show an improved therapeutic profile relative to existing APDs, in terms of improved clinical efficacy and reduced side effect profile.

The present inventors have observed that some existing agents used in the treatment of schizophrenia have affinity for 5-HT₇ and M₄ receptors, as well as many other receptors. There is however no suggestion in the art that the activity of the agents is due to a combination of their affinity and/or activity on the 5-HT₇ and M₄ receptors. These agents fall under the general grouping of bisorylazepines and in order to avoid any accidental anticipation by these compounds, such compounds are not encompassed by the present invention.

In addition, schizophrenic patients show marked deficits in cognitive tests, and this is thought to contribute to their inability to lead a relatively normal life. Since there is evidence that M₄ muscarinic agonists should act as cognitive enhancers (Jerusalinsky et al., 1998), a drug with substantial M₄ agonist activity may also be effective against the cognitive impairment characteristic of the disease.

Hence, the present inventors sought to demonstrate the potential therapeutic efficacy of a pharmacological agent combining selectivity versus other receptors with serotonin 5-HT₇ antagonist activity and muscarinic M₄ agonist activity.

The compounds with properties according to the present invention may be provided as pharmaceutical formulations wherein the compound or compounds is/are admixed with a pharmaceutically acceptable carrier (e.g. binder, corrective, corrigent, disintegrator, emulsion, excipient), diluent or solibilizer to give a pharmaceutical composition by a conventional manner, which is formulated into, for example, a tablet, capsule, granule, powder, syrup, suspension, solution, injection, infusion, deposit agent, suppository. Administration may be for example orally or parenterally.

When the tablets are used for oral administration, typically used carriers include sucrose, lactose, mannitol, maltitol, dextran, corn starch, typical lublicants such as magnesium stearate, preservatives such as paraben, sorbin, antioxidants such as ascorbic acid, α-tocopherol, cystein, disintegrators or binders. When administered orally as capsules, effective diluents include lactose and dry corn starch. A liquid for oral use includes syrup, suspension, solution and emulsion, which may contain a typical inert diluent used in this field, such as water. In addition, sweeteners or flavors may be contained.

In the case of parenteral administration such as subcutaneous injection, intraveneous injection, intramuscular injection, intraperitoneal injection or infusion, the pH of the active ingredient solution may be appropriately adequately adjusted, bufferized or sterilized. Examples of usable vehicle or solvent include distilled water, Ringer water and isotonic brine. For intraveneous use, the total concentration of solute is adjusted to make the solution isotonic.

Suppositories may be prepared by admixing the compounds of the present invention with a suitable nonirritative excipient such as those that are solid at normal temperature but become liquid at the temperature in the intestine and melt in rectum, such as cocoa butter and polyethylene glycols to release the active ingredient.

The dose can be determined depending on age, body weight, administration time, administration method, combination of drugs, the level of condition for which a patient is undergoing therapy, and other factors. While the daily dose may vary depending on the conditions and body weight of patients, the species of active ingredient, and administration route, in the case of oral use, the daily dose is about 0.1 mg-100 mg/person/day, preferably 0.5 mg-30 mg/person/day. In the case of parenteral use, the daily dose is desirably 0.1 mg-50 mg/person/day, preferably 0.1 mg-30 mg/person/day for subsutaneous injection, intraveneous injection, intramuscular injection and intrarectal administration.

Accordingly the agents and compounds according to the first and second aspects of the present invention, may be used in a method for treating psychotic disorders, for example schizophrenia for example the positive and/or negative symptoms of schizophrenia, and/or the cognitive deficits of schizophrenia, and/or bipolar disorder.

The present invention accordingly provides agents with properties according to the present invention and compounds represented by formulae (I), (II), (III) and (IV) for use in medicine or therapy.

According to a fourth aspect of the present invention, there is provided use of the agents with properties according to the present invention and compounds represented by formulae (I), (II), (III) and (IV) for the preparation of a medicament for the treatment of psychotic disorders, for example, schizophrenia e.g. the positive and/or negative symptoms of schizophrenia and/or the cognitive deficits of schizophrenia, and/or bipolar disorder.

According to a fifth aspect of the present invention, there is provided a method of identifying an agent having the properties according to the present invention comprising the steps of:
a) providing an agent to be tested;
b) subjecting said agent to one or more test procedures to identify 5-HT₇ receptor antagonist activity and muscarinic M₄ receptor agonist activity of said agent;
   wherein the desired agent is considered to have been identified when said agent provides a 5-HT₇ receptor antagonist activity and a muscarinic M₄ receptor agonist activity.

Desirably, the method further includes the step of subjecting the agent to a test procedure to identify low dopaminergic D₂ receptor affinity.

More preferably the agent is generally more selective than existing antischizophrenic and/or anti-bipolar disorder drugs. That is the agent has less affinity for other receptors than existing antischizophrenic and/or anti-bipolar disorder drugs.

Thus, the method may further comprise the step of subjecting the agent to a procedure to detect affinity for other receptors namely adrenergic α₁, α₂; histaminergic H₁, H₂, H₃; dopaminergic D₁, D₂, D₃, D₄, D₅; muscarinic cholinergic M₁, M₂, M₃, M₄, M₅; serotoneric 5-HT_{1A}, 5-HT_{2A}, 5-HT_{2B}, 5-HT_{2C}, 5-HT₃, 5-HT₆, 5-HT₇ and selecting agents which display affinity for less than 75% of said receptors, preferably less then 50% of said receptors.

The present invention will now be described by way of example with reference to the following experimental section and drawings in which:

FIG. 1 is a representation of a full treatment paradigm of chronic PCP rat model with PTAC ((5R,6R)6-(3-propylthio-1,2,5-thiadiazol-4-yl)-1-azabicyclo(3.2.1)octane) and SB258741 (R-(+)-1-(toluene-3-sulfonyl)-2-[2-(4-methylpiperidin-1-yl)ethyl]pyrrolidine, CNS Drug Rev., 2002 Spring; 8(1):90-100);

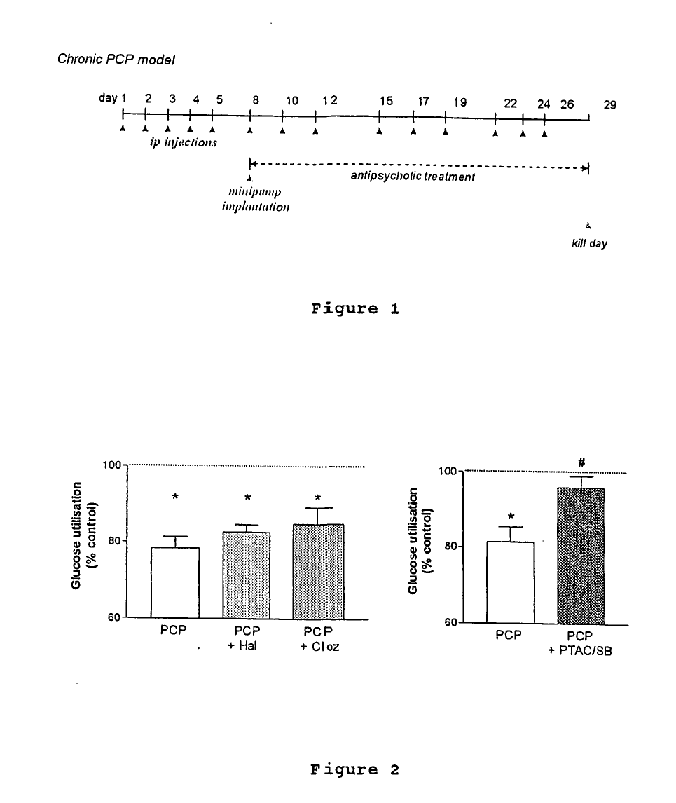

FIG. 2 shows the effect of haloperidol (Hal), clozapine (cloz) or the experimental serominic combination—PTAC+SB258741 (PTAC/SB)—on chronic PCP-induced hypofrontality;

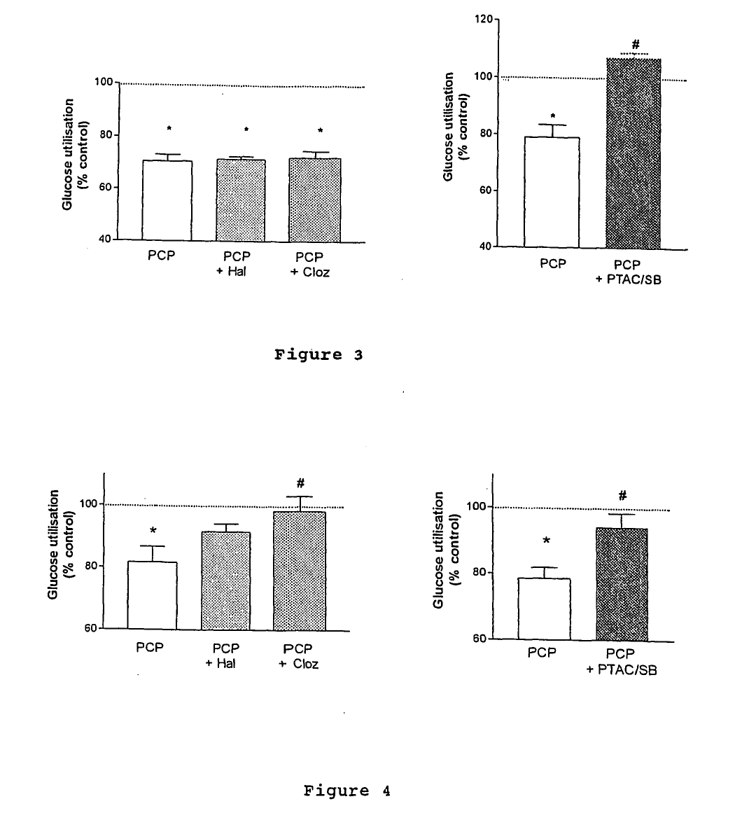

FIG. 3 relates to the reticular thalamic metabolic activity and shows the effect of haloperidol (Hal), clozapine (cloz) or the experimental serominic combination—PTAC+SB258741 (PTAC/SB)—on chronic PCP-induced hypoactivity;

FIG. 4 relates to the auditory structure metabolic activity and shows the effect of haloperidol (Hal), clozapine (cloz) or the experimental serominic combination—PTAC+SB258741 (PTAC/SB)—on chronic PCP-induced hypoactivity.

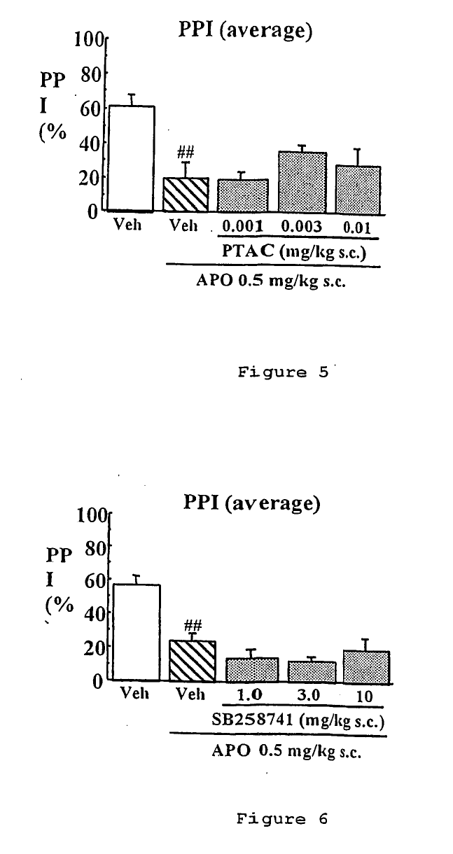

FIG. 5—Effects of PTAC alone on apomorphine-induced deficits in PPI in rats. Values represent mean±SEM. ##p<0.01 compared to Vehicle+APO group and PTAC treated groups (Dunnett's test). n=7.PPI (average) means that PPI collapsed across all three prepulse intensities (73, 75 and 80 dB).

FIG. 6—Effects of SB258741 alone on apomorphine-induced deficits in PPI in rats. Values represent mean±SEM. ##p<0.01 compared to Vehicle+Vehicle group (t-test). There are no significant difference between Vehicle⁺APO group and SB258741 treated groups (Dunnett's test). n=7.

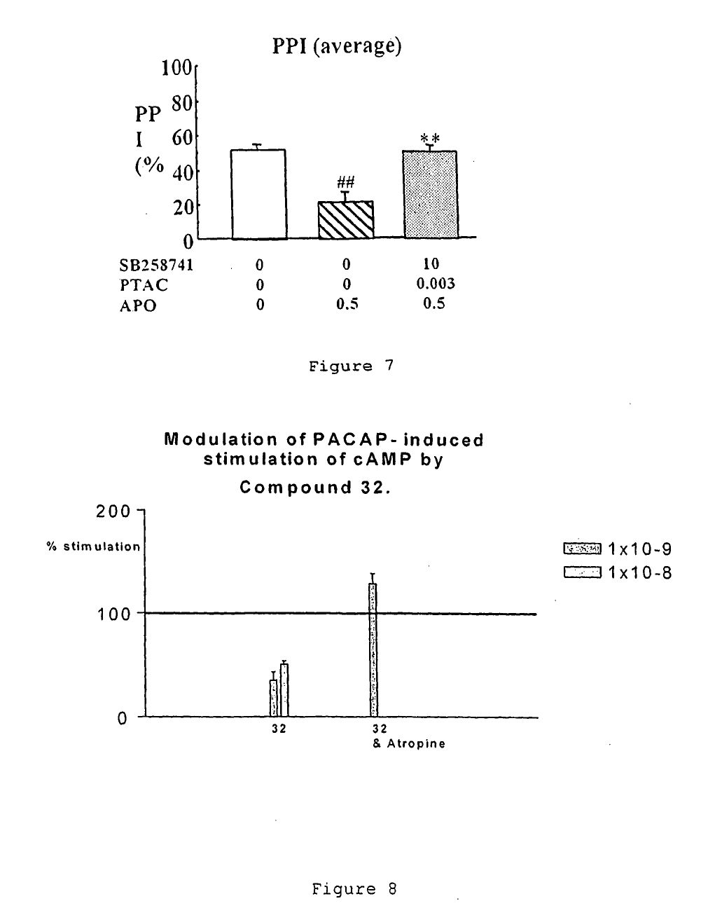

FIG. 7—Synergisitic effect of PTAC and SB258741 on apomorphine-induced deficits in PPI in rats. Values represent mean±SEM. ##p<0.01 compared to Vehicle+Vehicle+Vehicle group (t-test). **p<0.01 compared to Vehicle+Vehicle+APO group (t-test). n=7.

FIG. 8 shows the modulation of PACAP-induced stimulation of cAMP by the compound 32.

Example 1 gives the various methods and results of testing for compound efficacy in the treatment of schizophrenia;

Example 2 gives the various methods and results of screening for binding affinity of the compounds of the present invention and in vivo testing;

Example 3 describes several examples for the preparation of the compounds of the present invention.

### EXAMPLE 1

In Vivo Activity:

To test the hypothesis that a serominic compound would show efficacy in the treatment of schizophrenia, we exploited our recent discovery of an animal model of schizophrenia that mimics the neurochemical and metabolic dysfunction in the brains of patients with schizophrenia (Cochran et al., 2003).

Schizophrenic patients show reduced metabolic activity in the prefrontal cortex, auditory system and hippocampus, along with reduced levels of expression of parvalbumin within inhibitory interneurones of the prefrontal cortex. The hypometabolism in the prefrontal cortex is not restored to normal by typical APDs, or by atypical APDs such as clozapine, although the hypometabolism in the auditory system is thought to be improved by both typical and atypical APDs (Schroeder et al., 1994; Andreasen et al., 1992 and Potkin et al., 1994). We have previously reported (Cochran et al., 2003) that these deficits observed in schizophrenic patients are reproduced in rats treated chronically with phencyclidine (PCP)—a drug known to cause schizophrenic symptoms when administered chronically in humans. We have also observed that, in parallel with the clinical observations, the prefrontal cortex hypometabolism in PCP-treated rats is not attenuated by the representative atypical and typical antipsychotic drugs clozapine or haloperidol (Cochran et al., 2003), whereas the hypometabolism in the auditory system is restored towards normal levels by both haloperidol and clozapine. Thus evidence that a serominic compound could restore the prefrontal cortex hypometabolism in PCP-treated rats towards normal levels would indicate that a serominic compound would be more effective than currently available antipsychotic drugs for the treatment of schizophrenia. (5R,6R)6-(3-propylthio-1,2,5-thiadiazol-4-yl)-1-azabicyclo(3.2.1)octane.

When the M₄ muscarinic partial agonist (PTAC) (Calbiochem Biochemicals) was administered chronically in combination with the 5-HT₇ antagonist SB258741, the drug combination was found to attenuate the hypometabolism in the prefrontal cortex, and thus demonstrate efficacy superior not only to haloperidol, but also to clozapine. A similar effect was observed in the reticular thalamus, which is a brain region functionally connected with the prefrontal cortex and involved in the regulation of its activity. In addition, the hypometabolism in the auditory system was also restored to normal by the M₄ agonist/5-HT₇ antagonist combination. Thus, the combination of M₄ agonist/5-HT₇ antagonist appears to exert profound antipsychotic activity, as assessed by these markers, in the absence of any D₂ affinity.

Experimental Procedure

Male hooded Long Evans rats (180-220 g) were randomly allocated to one of the following treatment groups: vehicle/vehicle, PCP/vehicle, and PCP/SB258741+PTAC. The first drug (PCP or vehicle) was administered by i.p injection and the second drug combination (SB258741+PTAC) was delivered via osmotic minipump which was implanted under halothane anaesthetic on day 8 of the YRING PCP model. See WO01/75440. The doses of drug used were 2.58 mg/kg PCP, vehicle (sterile saline), 0.1 mg/kg/day PTAC together with SB258741 20 mg/kg/day. The full treatment paradigm of the chronic PCP model is shown in FIG. 1.

On the day of the 2-DG procedure, the animals were prepared according to the method of Crane and Porrino, (1989). The brains were sectioned and exposed to X-ray film and LCGU measurements were calculated using the MCID 5 densitometry system. The results were analysed using a one way ANOVA followed by LSD post hoc test where appropriate for each discrete brain region. Statistical significance was defined as p<0.05.

The rats treated chronically with the M₄ agonist/5-HT₇ antagonist combination did not show any overt evidence of side-effects.

LCGU within Cortical Regions

The effect of PTAC and SB258741 (the serominic combination) on LCGU within cortical brain regions is shown in table 1.1. The only non-auditory cortical brain region which showed a significant metabolic hypofunction induced by PCP was in the prefrontal cortex. Within the prelimbic region of the prefrontal cortex, a significant decrease in LCGU following chronic PCP compared to controls was observed in layer I (19%) and layers II and III (25%). Layers V&VI was just outside statistical significance. When SB258741+PTAC were administered in conjunction with PCP they reversed the PCP-induced hypofunction back to control levels (see table 1.1). The medial orbital cortex also displayed a significant decrease in LCGU following chronic PCP treatment compared to control animals within layer I (16%), layers II&III (19%) and layers V and VI (16%). No other cortical brain region showed any significant alterations in LCGU following any combination of drug treatment.

LCGU within Auditory Structures

Table 1.2 shows the effect of the serominic combination given in combination with the YRING PCP Model on LCGU in auditory brain structures. PCP treatment induced a metabolic hypofunction within a few structures of the auditory system. Within the ventral lateral lemniscus, the ventral cochlear nucleus and the primary auditory cortex chronic PCP treatment significantly reduced LCGU (26%, 21% and 25% respectively). In all these three auditory structures the serominic combination reversed the PCP-induced hypofunction

LCGU within Thalamic Nuclei

The effect of the serominic given in combination with the YRING PCP Model on LCGU within thalamic brain regions is shown in table 1.3. The only thalamic nuclei which displayed a metabolic hypofunction with PCP was the reticular thalamus. Within the dorsal region of the reticular thalamus LCGU was significantly decreased by 25% and in the ventral reticular thalamus the PCP-induced decrease was 21%. In both regions of the reticular thalamus the serominic combination completely reversed the PCP-induced hypofunction.

TABLE 1.1
The effect of chronic SB258741 + PTAC treatment on chronic PCP
induced changes in LCGU within cortical region. All data expressed
as mean LCGU (μmol/100 g/min) ± SEM (n = 5-7). *signifies
p < 0.05 compared to vehicle—vehicle treated animals, #signifies
p < 0.05 compared to PCP-vehicle treated animals. The abbreviations
used in the table are listed hereinafter.

LCGU (μmol/100 g/min)

PCP

| | Vehicle | | Serominic |
|---|---|---|---|
| | vehicle | Vehicle | Combination |
| MO1 | 113 ± 4 | 95 ± 3* | 98 ± 4* |
| MO2 | 118 ± 7 | 95 ± 6* | 112 ± 5# |
| MO3 | 111 ± 8 | 93 ± 3* | 102 ± 5 |
| vO1 | 135 ± 8 | 153 ± 8 | 163 ± 3 |
| vO2 | 165 ± 8 | 168 ± 12 | 158 ± 10 |
| vO3 | 155 ± 8 | 151 ± 9 | 147 ± 8 |
| lO1 | 153 ± 7 | 150 ± 11 | 162 ± 5 |
| lO2 | 158 ± 8 | 155 ± 10 | 138 ± 7 |
| lO3 | 148 ± 6 | 138 ± 7 | 138 ± 7 |
| PrL1 | 129 ± 1 | 105 ± 5* | 124 ± 4# |
| PrL2 | 144 ± 6 | 108 ± 4* | 145 ± 2# |
| PrL3 | 148 ± 7 | 128 ± 8 | 143 ± 7 |
| IL1 | 97 ± 5 | 98 ± 6 | 100 ± 5 |
| IL2 | 99 ± 5 | 102 ± 7 | 99 ± 5 |
| IL3 | 96 ± 5 | 96 ± 7 | 97 ± 6 |
| M1 | 143 ± 9 | 128 ± 7 | 132 ± 6 |
| M2 | 124 ± 9 | 117 ± 4 | 119 ± 2 |
| Cg1 | 127 ± 8 | 123 ± 5 | 124 ± 6 |
| Cg2 | 138 ± 8 | 135 ± 6 | 132 ± 4 |
| Cg3 | 117 ± 7 | 119 ± 6 | 108 ± 3 |
| Pir | 153 ± 7 | 137 ± 7 | 139 ± 6 |
| I | 92 ± 6 | 86 ± 7 | 84 ± 5 |
| RS1 | 117 ± 7 | 116 ± 11 | 119 ± 2 |
| RS2 | 116 ± 7 | 120 ± 11 | 120 ± 5 |
| RS3 | 103 ± 7 | 110 ± 11 | 107 ± 4 |
| Ent1 | 79 ± 6 | 81 ± 4 | 83 ± 5 |
| Ent2 | 73 ± 5 | 77 ± 4 | 73 ± 5 |
| Ent3 | 69 ± 4 | 72 ± 5 | 63 ± 8 |

TABLE 1.2
The effect of chronic SB258741 + PTAC treatment on chronic PCP
induced changes in LCGU within auditory structures. All data expressed
as mean LCGU (μmol/100 g/min) ± SEM (n = 5-7). *signifies
p < 0.05 compared to vehicle—vehicle treated animals, #signifies
p < 0.05 compared to PCP-vehicle treated animals. Appendix 1 details
the abbreviations used in the table.

| LCGU (μmol/100 g/min) | |
|---|---|
| Vehicle | PCP |

| | Vehicle | Vehicle | Serominic |
|---|---|---|---|
| AudCx1 | 177 ± 7 | 133 ± 4* | 164 ± 8# |
| AudCx2 | 140 ± 16 | 123 ± 3 | 131 ± 6 |
| VisCx1 | 147 ± 6 | 129 ± 11 | 133 ± 9 |
| VisCx2 | 129 ± 8 | 115 ± 8 | 117 ± 9 |
| ILL | 113 ± 6 | 94 ± 6 | 110 ± 6 |
| DLL | 105 ± 4 | 87 ± 3 | 112 ± 5 |
| VLL | 121 ± 7 | 95 ± 4* | 114 ± 5# |
| VCP | 129 ± 8 | 95 ± 4* | 124 ± 5# |

TABLE 1.3
The effect of chronic SB258741 + PTAC treatment on chronic PCP
induced changes in LCGU within thalamic nuclei. All data expressed
as mean LCGU (μmol/100 g/min) ± SEM (n = 5-7). *signifies
p < 0.05 compared to vehicle—vehicle treated animals, #signifies
p < 0.05 compared to PCP-vehicle treated animals. Appendix 1
details the abbreviations used in the table.

| LCGU (μmol/100 g/min) | |
|---|---|
| Vehicle | PCP |

| | Vehicle | Vehicle | Serominic |
|---|---|---|---|
| AV | 161 ± 8 | 145 ± 10 | 148 ± 3 |
| AM | 144 ± 8 | 138 ± 8 | 139 ± 5 |
| Rt dorsal | 107 ± 6 | 80 ± 5* | 109 ± 2# |
| Rt ventral | 115 ± 7 | 91 ± 5* | 120 ± 2# |
| G | 155 ± 9 | 142 ± 11 | 140 ± 5 |
| Re | 107 ± 8 | 118 ± 7 | 115 ± 4 |
| Rh | 106 ± 3 | 102 ± 6 | 110 ± 3 |
| VL | 119 ± 4 | 121 ± 11 | 116 ± 3 |
| VM | 137 ± 10 | 137 ± 13 | 134 ± 4 |
| PV | 87 ± 5 | 88 ± 6 | 83 ± 2 |
| MD | 133 ± 10 | 131 ± 8 | 133 ± 4 |
| CM | 117 ± 6 | 110 ± 6 | 113 ± 2 |
| CL | 122 ± 8 | 129 ± 10 | 127 ± 2 |
| IM | 108 ± 4 | 109 ± 6 | 112 ± 4 |

This study has shown that the chronic PCP-induced metabolic hypofunction in the prelimbic region of the prefrontal cortex is completely reversed back to control levels when administered with chronic PCP and the serominic combination. This is of great interest as we have shown previously (Cochran et al., 2003) that both clozapine and haloperidol failed to reverse this PCP-induced metabolic hypofunction in the prelimbic region of the prefrontal cortex.

Local glucose utilisation was measured in the prelimbic area of the prefrontal cortex after chronic treatment with PCP alone or with the antipsychotic drugs. Note that the reduced metabolic activity caused by PCP (*p<0.05 vs control) is restored to normal values by the serominic combination (#p<0.05 vs PCP alone) but not by haloperidol or clozapine.

Thus, the combination of M₄ agonist/5-HT₇ antagonist appears to exert profound antipsychotic activity, as assessed by these markers, in the absence of any D₂ antagonist activity. This provides dramatic evidence that an agent with serominic properties is likely to be—markedly superior to any of the currently-available antipsychotic agents.

This inability of haloperidol and clozapine to modulate the hypofrontality is consistent with data from clinical studies where similar results are obtained in medicated and unmediated patients (Schroeder et al., 1994; Andreasen et al., 1992 and Potkin et al., 1994). The prefrontal cortex is involved in working memory, attention and cognitive flexibility and has been implicated in the cognitive dysfunction observed in schizophrenic patients. Also this hypofunction has been correlated to the intensity of negative and cognitive dysfunction of schizophrenia (Wolkin et al., 1992: Schroder et al., 1995). There is conflicting evidence that clozapine and other new atypical antipsychotics are effective in treating these symptoms of the disease, but it is generally accepted that the negative symptoms and cognitive impairments seen in schizophrenia have proved very difficult to treat to date (Goldberg et al., 1993). In this study we have shown that the serominic combination can reverse the PCP-induced hypofunction in the prefrontal cortex.

In the reticular nucleus of the thalamus the PCP-induced metabolic hypofunction is restored to control levels when the serominic combination is administered (FIG. 3). Previously we have shown that both clozapine and haloperidol failed to reverse the PCP-induced hypofunction in the reticular nucleus of the thalamus (Cochran et al., 2003).

Local glucose utilisation was measured in the ventral reticular thalamic nucleus after chronic treatment with PCP alone or with the antipsychotic drugs. Note that the reduced metabolic activity caused by PCP (*p<0.05 vs control) is restored to normal values by the serominic combination (#p<0.05 vs PCP alone) but not by haloperidol or clozapine.

The mechanism by which serominic is reversing the PCP-induced metabolic hypofunction in the reticular thalamus is postulated to be through a 5-HT₇ receptor mediated mechanism since 5-HT₇ receptors are concentrated in thalamic areas.

The fact that the serominic combination can reverse the PCP-induced hypofunction in the dorsal and ventral parts of the reticular thalamus is again of much interest as it suggests that the serominic may be beneficial in treating the positive symptom of the disease (poor filtering of irrelevant information) and also indirectly in treating the negative symptoms and cognitive deficits as the reticular thalamus has reciprocal projections to the prefrontal cortex. Once again, the serominic combination shows superior efficacy to current APDs.

In selected auditory brain structures (ventral lateral lemniscus, ventral cochlear nucleus and in the primary auditory cortex) chronic PCP treatment caused a significant hypofunction. Previously, we reported that both clozapine and haloperidol reversed the PCP-induced hypofunction in these auditory structures (Cochran et al., 2003), consistent with their efficacy against positive symptoms such as auditory hallucinations. This study shows that the serominic is also effective in reversing the metabolic hypofunction within these auditory structures.

Local glucose utilisation was measured in the ventral lateral lemniscus after chronic treatment with PCP alone or with the antipsychotic drugs. Note that the reduced metabolic activity caused by PCP (*p<0.05 vs control) is restored to normal values by clozapine or the serominic combination (#p<0.05 vs PCP alone) but haloperidol only partially restores the hypofunction. Similar effects were observed in the auditory cortex and other auditory structures.

Decreased metabolism of the temporal lobe (auditory cortex and hippocampus) have been directly correlated with the positive symptoms of the disease (Buchsbaum et al, 1996; Klemm et al., 1996). Therefore this study shows that the serominic is effective in reversing the metabolic hypofunction within these auditory structures, which are associated with hallucinations (positive symptom of the disease). Therefore it appears that a serominic agent will behave in a similar way to clozapine and haloperidol in ameliorating the positive symptoms of the disease.

These results imply that a serominic may be beneficial in treating both the negative symptoms and cognitive impairment which to date have proved very difficult to treat, as well as being effective in treating the positive symptoms of the disease.

The startle reaction to a strong acoustic stimulus is reduced by the prior presentation of a weak stimulus. This reduction, termed prepulse inhibition (PPI), has been used as a measure of sensorimotor gating and significantly diminished in schizophrenic patients (Braff et al., 1978). In rats, the disruption of PPI by apomorphine is reversed by the administration of antipsychotics with potency correlating well with clinically effective dosages of each drug. Thus, the disruption of PPI by apomorphine is a valid animal model for some aspects of schizophrenia (Swerdlow et al., 1994).

Methods

Male Wistar rats (Japan SLC Inc., Shizuoka, Japan) were used. They were housed in a light, humidity and temperature controlled environment maintained on a 12-hour/12-hour light/dark schedule (light on at 7 am) with food and water provided ad libitum. Four startle chambers (SR-LAB, San Diego Instruments, San Diego, Calif.) were placed in a sound-attenuated room. Each chamber consisted of Plexiglas cylinder 8.8 cm in diameter resting on a 12.7×20.3 cm Plexiglas stand. Acoustic stimuli and background noise were presented via a Supertweeter mounted 24 cm above the Plexiglas cylinder. Startle magnitude was detected and recorded by a microcomputer and interface assembly (San Diego Instruments) as transduced cylinder movement via a piezoelectric device mounted below the Plexiglas stand.

One day before drug testing, all rats were exposed to a “matching” startle session. Data from this session were used to assign rats to balanced groups according to their startle responses. On the drug testing day, rats were treated with vehicle (sterile saline) or drug (PTAC and/or SB258741) subcutaneously 25 minutes prior to apomorphine (0.5 mg/kg, s.c.) treatments. Immediately after apomorphine treatments, rats were placed into the startle chamber and a test session was started. Each session was approximately 20 minutes and consisted of 5 minutes of 70-dB background followed by five trial types, PULSE ALONE trial: a 120-dB 50 ms noise burst, PREPULSE trials which consisted of 20 ms noise bursts 3, 5, 10 dB above 70-dB background noise followed 100 ms later by a 120-dB 40 ms noise burst, NOSTIM trial: 100 ms of response was recorded during periods where no stimulus was presented. Each trial presented in pseudorandom order every 15 seconds for a total 60 trials (12 trials each). The percentage PPI was defined as 100-[(startle amplitude on PREPULSE trial/startle amplitude on PULSE ALONE trial)×100].

Results

Apomorphine (0.5 mg/kg, s.c.) significantly reduced the PPI (FIG. 1,2,3). Neither PTAC nor SB25871 alone affect the disruption induced by apomorphine (FIGS. 1 and 2, respectively). PTAC combined with SB25871 restored the apomorphine-induced disruption of PPI (FIG. 3).

The agents with properties according to the present invention are useful as a novel type of antipsychotic agent which are effective for both the positive and negative symptoms of schizophrenia, and which may cause less side effects of extrapyramidal motor disorder and the like and which may cause less serious side effects such as agranulocytosis and the like.

### EXAMPLE 2

The compounds of the present invention were screened for binding affinity using membranes containing stably expressed human M₄ muscarinic receptors or human 5-HT₇ receptors.

M₄ Assay:

Total volume 200 μl/well. Membrane concentration—human M₄ membranes (NEN)—8 μg/ml; ³H-NMS 0.25 nM; Sample conc. 10 nM-300 μM; Atropine Displacement Curve—0.3 nM-1 μM.

The plates are incubated at 20° C. for 60 minutes in the dark to avoid any photo degradation. Membranes are harvested by rapid filtration using a vacuum manifold under 700 mbar pressure. The plates are washed 3 times with 200 ul per well of ice-cold wash buffer. Plates are dried at 40° C. for 1 hour, 100 μl scintillation fluid is added to each well and cpm determined using a Microbeta scintillation counter.

5-HT₇ Assay:

Total volume 200 μl/well. Membrane concentration—human 5-HT₇ membrane (purchased from Euroscreen)—6 μg/ml; ³H-5CT 0.5 nM; Sample conc. 10 nM-300 μM; 8OH-DPAT Displacement Curve—1 nM-3 μM. The plates are incubated at 20° C. for 120 minutes in the dark to avoid any photo degradation. Membranes are harvested by rapid filtration using a vacuum manifold under 700 mbar pressure. The plates are washed 3 times with 200 ul/well of ice-cold wash buffer. Plates are dried at 40° C. for 1 hour (Higher CPMs are obtained when the filters are dried) 100 μl scintillation fluid is added to each well and cpm determined using a Microbeta scintillation counter.

D₂ Assay:

Total volume 200 μl/well. Membrane concentration—human D₂ membrane (purchased from Euroscreen)—10 μg/ml; ³H-spiperone 0.5 nM; Sample conc. 10 nM-300 μM; Haloperidol Displacement Curve—1 nM-10 μM. The plates are incubated at 25° C. for 60 minutes in the dark to avoid any photo degradation.

Membranes are harvested by rapid filtration using a vacuum manifold under 700 mbar pressure. The plates are washed 3 times with 200 ul/well of ice-cold wash buffer. Plates are dried at 40° C. for 1 hour (Higher CPMs are obtained when the filters are dried). 100 μl scintillation fluid is added to each well and cpm determined using a Microbeta scintillation counter.

The following are examples showing data for Compounds 32 and 34

| | Ki | Ki | Ki |
|---|---|---|---|
| | (5-HT₇) | (M₄) | (D₂) |
| Compound | (μM) | (μM) | (μM) |
| Compound 32 | 0.4 | 0.32 | >300 |
| Compound 34 | 2.7 | 2.8 | >300 |

Efficacy (cAMP): Homogenate Assay for c-AMP Production
Methods:

N1E-115 cells were harvested by scraping and placed in Ribolyser tubes on dry ice. Ice cold buffer (0.5 ml) containing 50 mM Tris HCl, 0.4 mM EDTA and 0.4 mM EGTA (pH 7.4) was added to the tubes. The tubes were then placed in a Ribolyser and shaken at 4 g for 20 sec. The homogenate was then transferred to eppendorf tubes and was then centrifuged at 19,700 g for 30 min at 4° C. The pellet was resuspended in ice cold 50 mM Tris HCl (pH7.4) at a concentration of 50 mg ml⁻¹ wet weight of tissue. The homogenate was stored in aliquots at −70° C. Protein concentrations of the homogenates were determined using a Bio-Rad protein determination kit. The assay was carried out in a final assay volume of 120 μl containing 50 mM Tris HCl (pH7.4), 5 mM MgCl₂ 50 μM GTP, 200 μM ATP, 120 μM sucrose, 0.4 mM EDTA, 0.4 mM EGTA, 200 μM ascorbic acid, 20 μM papaverine, 200 μM rolipram, 10 μM vinpocetine, 10 mM phosphocreatinine, 0.4 mM DTT, 100 nM WAY 100635, 1 μM propranolol, 36 μg bacitracin, 4.8 U creatine phosphokinase, 3.6 KIU aprotinin. Homogenate (1 mgml⁻¹) was preincubated with the test compound in ice cold assay buffer for 10 min. PACAP (0.1 nM) was then added to the tubes. The tubes were then incubated at 30° C. for 20 min and then at 99° C. for 5 min. Levels of c-AMP in the tubes were measured using the Amersham Pharmacia Biotech Biotrak c-AMP enzymeimmunoassay kit.

Results:

Mouse N1E-115 cells express a pure population of M₄ muscarinic receptors, negatively coupled to c-AMP levels. Known muscarinic agonists with activity at M₄ receptors, including oxotremorine and acetylcholine, showed the ability to reduce c-AMP levels.

Compounds of this series also showed similar agonist activity: an example is shown for 32 in FIG. 1.

Compound 32 was able to reduce cAMP levels, and the effect was blocked by the muscarinic antagonist atropine.

In Vivo Activity:

To test the hypothesis that these compounds would show efficacy in the treatment of schizophrenia, we tested their inhibitory effect on a standard test for antipsychotic activity—amphetamine-induced hyper-activity in rats.

Methods

Male Long Evans rats (190-280 g) in each group of five were used.

Amphetamine (1.0 mg/kg i.p.) was dissolved in saline, and test compounds were dissolved or suspended in 0.5% hydroxypropylmethylcellulose (HPMC) solution. All the test compounds were injected intraperitoneally in a volume of 0.1 ml/100 g, and control rats were treated with the respective vehicle.

The plastic open-field box (40×40×40(H) cm) was used to measure the locomotor activity of rats. The locomotor activity was expressed as the number of line crossings marked on the floor of the test box at 20 cm square. Individual rats were placed in the test box just after the injection of amphetamine, and were allowed to habituate there for 10 min. The line crossings were counted over 15 min thereafter. The behavioural observation was conducted on two rats simultaneously using two test boxes.

Test compounds were pretreated 30 min before the injection of amphetamine.

Results

Compound 32 suppressed the hyperactivity in a dose-dependent manner, of which ED50 value was estimated as 8.1 (95% confidence limits; 4.4-15) mg/kg, i.p. (Table 2). Table 2 Effect of Compound 32 on amphetamine-induced hyperactivity in rats

| Dose (mg/kg, i.p.) | Line Crossings (mean ± S.E.) |
|---|---|
| 0 | 111.6 ± 6.4 |
| (amphetamine | |
| control) | |
| 1 | 98.8 ± 7.3 |
| 3 | 89.6 ± 13.3 |
| 10 | 45.6 ± 8.3** |

The compounds of formula (I) of the present invention are useful as a novel type of the antipsychotic agents which are effective for both the positive and negative symptoms of schizophrenia, which causes less side effects of extrapyramidal motor disorder and the like and which causes less serious side effects such as agranulocytosis and the like.

### EXAMPLE 3

The following are some examples for the preparation of the compounds of the present invention:

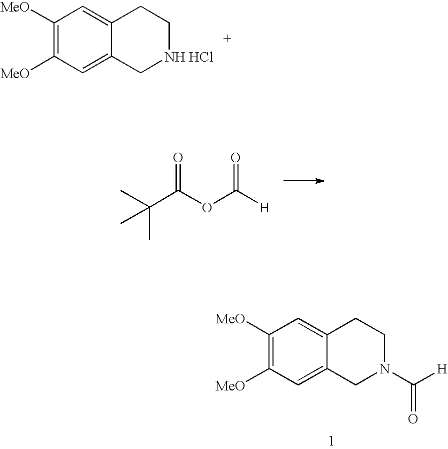

Trimethylacetic formic anhydride (5.3 g, 40.77 mmol) (E. J. Vlietstra et al., Journal of the Royal Netherlands Chemical Society, 101/12, 1982, 460-462) was added to a solution of 6,7-dimethoxy-1,2,3,4-tetrahydroisoquinoline hydrochloride (9.36 g, 40.77 mmol) in dry dichloromethane (40 mL) cooled in an ice-water bath under nitrogen atmosphere, followed, dropwise, by dry triethylamine (4.95 g, 58.924 mmol). The mixture was stirred at room temperature for 1 h and was then diluted with dichloromethane, washed with dilute hydrochloric acid (2M), saturated sodium bicarbonate, the organic phase was dried over MgSO₄, filtered and concentrated. Purification by flash chromatography (pure EtOAc to dichloromethane/MeOH 95/5) afforded compound 1 (8.29 g, 92%) as a white solid consisting of two rotamers (major:minor ratio ca. 2:1) in ¹HNMR spectrum (all J values are quoted in Hertz). ¹H NMR (CDCl₃, 400 MHz): 2.78-2.85 (2H, m), 3.63 (major) and 3.78 (minor) [2H, 2×t, J 5.9 (major) and 6.1 (minor)], 3.86 (6H, s), 4.48 (minor) and 4.61 (major) (2H, 2×s), 6.58-6.63 (2H, m, ArH), 8.26 (minor) and 8.19 (major) (1H, 2×s).

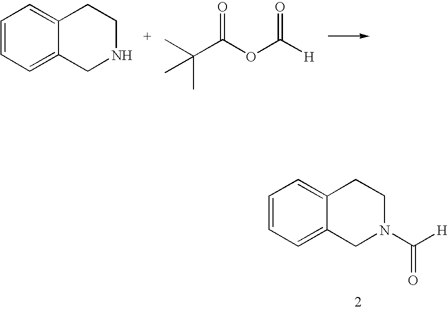

Trimethylacetic formic anhydride (3.12 g, 23.79 mmol) was added dropwise to a solution of 1,2,3,4-tetrahydroisoquinoline (2.9 g, 21.79 mmol) in chloroform (20 mL) cooled in an ice-water bath under nitrogen atmosphere. The mixture was stirred at room temperature for 1 h and then diluted with dichloromethane, washed with dilute hydrochloric acid (2M), saturated sodium bicarbonate, the organic phase was dried over MgSO₄, filtered and concentrated. Purification by flash chromatography (pure EtOAc to dichlormethane/MeOH 95/5) afforded compound 2 (2.98 g, 85%) as a pale yellow oil, consisting of two isomers (major:minor ratio, ca. 1.5:1) in ¹H NMR spectrum (CDCl₃, 400 MHz) ¹H NMR: 2.86-2.93(2H, m, ArCH₂), 3.65 (major) and 3.79 (minor) [2H, 2×t, J 5.9 (major), 6.1 (minor) CH₂N], 4.54 (minor) and 4.69 (major) (2H, 2×s, ArCH₂N), 7.09-7.23 (4H, m, ArH), 8.20 (major) and 8.25 (minor) (1H, s, CHO).

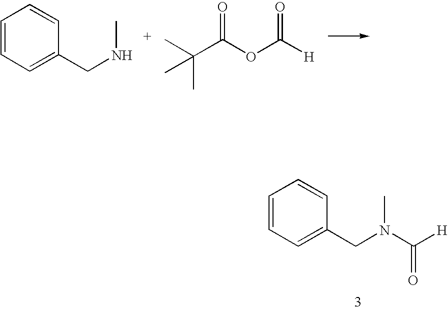

Trimethylacetic formic anhydride (3.55 g, 27.27 mmol) was added dropwise to a solution of N-methylbenzylamine (3.0 g, 24.79 mmol) in dry dichloromethane (20 mL) cooled in an ice-water bath under nitrogen atmosphere. The mixture was stirred at room temperature for 1 h and then diluted with dichloromethane, washed with dilute hydrochloric acid (2M), saturated sodium bicarbonate, the organic phase was dried over MgSO₄, filtered and concentrated. Purification by flash chromatography (pure EtOAc to dichloromethane/MeOH, 95/5) afforded compound 3 (3.1 g, 84%) as a pale yellow oil, consisting of two rotamers (major:minor ratio ca. 1.2/1) in ¹H NMR spectrum. ¹HNMR (CDCl₃, 400 MHz): 2.84 (major) and 2.90 (minor) (3H, 2×s, NMe), 4.45 (major) and 4.85 (minor) (2H, 2×s, NCH₂), 7.25-7.45 (5H, m, ArH), 8.22 (minor) and 8.34 (major) (1H, 2×s, CHO).

Preparation of Compound 4

A solution of potassium nitrate (50.5 g, 0.5 mol) in H₂SO₄ (200 mL) was added, via a dropping funnel, to a solution of 1-indanone (60 g, 0.454 mol) in concentrated sulfuric acid (500 mL) cooled in an ice-water bath at a speed to maintain an internal temperature below 15° C. After stirring at 0° C. for 1 h, the reaction mixture was poured into crushed ice and stirred for 30 min. The solid was filtered, washed with water, and air-dried. Purification by flash chromatography (toluene/EtOAc, 95/5) gave compound 4 (43.5 g, 54%) as a pale yellow solid. ¹H NMR (CDCl₃, 400 MHz): 2.81-2.85 (2H, m, CH₂), 3.28 (2H, t, J 6.1, CH₂), 7.67 (1H, d, J 8.4, ArH), 8.45 (1H, d, J 8.4, ArH), 8.56 (1H, s, ArH).

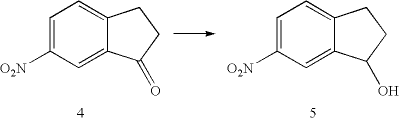

A solution of 4 (2.7 g, 15.254 mmol) in MeOH (50 mL) was cooled in an ice-water bath and sodium borohydride (580 mg, 15.254 mmol) was added in three portions. The reaction was continued at room temperature for 30 min, quenched by adding hydrochloric acid (2M, 30 mL). Most of the methanol was removed by rotavapor, the residue was diluted with water, extracted with dichloromethane, the organic phase was dried over MgSO₄, filtered and concentrated to provide crude alcohol 5 as a brown solid. The product was used in the next reaction without further purification. ¹H NMR (CDCl₃, 400 MHz): 2.00-2.08 (1H, m, CH₂), 2.44 (1H, broad, OH), 2.56-2.63 (1H, m, CH₂), 2.85-2.94 (1H, m, CH₂), 3.08-3.16 (1H, m, CH₂), 5.30-5.31 (1H, m, CHO), 7.36 (1H, d, J 8.3, ArH), 8.11 (1H, dd, J 8.3, 2.0, ArH), 8.22 (1H, d, J 2.0, ArH).

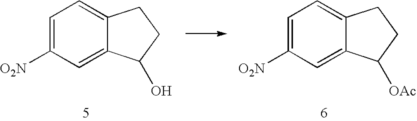

To the solution of crude 5 in pyridine (20 mL) under nitrogen was added acetic anhydride (6 mL) at 0° C. and the mixture was stirred at room temperature overnight. The mixture was poured into water, extracted with diethyl ether, the organic phase was washed with hydrochloric acid (2M), dried over MgSO₄, filtered and concentrated. Purification by flash chromatography (petroleum ether/EtOAc, 75/25) gave compound 6 (3.23 g, 95% for two steps) as a slightly yellow oil. ¹H NMR (CDCl₃, 400 MHz): 2.10 (3H, s, Ac), 2.14-2.23 (1H, m, CH₂), 2.57-2.66 (1H, m, CH₂), 2.93-3.01 (1H, m, CH₂), 3.14-3.23 (1H, m, CH₂), 6.19-6.23(1H, m, CHO), 7.41 (1H, d, J 8.3 Hz, ArH), 8.18 (1H, d, J 8.3 Hz, ArH), 8.24 (1H, s, ArH).

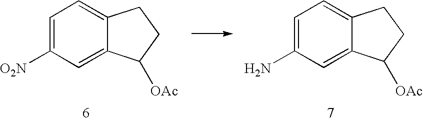

A solution of 6 (1.0 g, 4.52 mmol) in MeOH (10 mL) was subjected to hydrogenation at atmospheric pressure with Pd/C as catalyst. The reaction was followed carefully by TLC and was stopped when most of the starting material was consumed. The mixture was filtered through kieselguhr and was concentrated. Purification by flash chromatography (petroleum ether/EtOAc, 60/40) gave compound 7 (460 mg, 53%) as a pale brown oil. ¹H NMR (CDCl₃, 400 MHz): 2.01-2.11 (4H, m, Ac+CH₂), 2.41-2.51 (1H, m, CH₂), 2.72-2.79 (1H, m, CH₂), 2.95-3.03 (1H, m, CH₂), 3.71 (2H, broad, NH₂), 6.11-6.14 (1H, m, ArCH), 6.62 (1H, dd, J 8.4, 2.2, ArH), 5.75 (1H, d, J 2.2, ArH), 7.04 (1H, d, J 8.4, ArH).

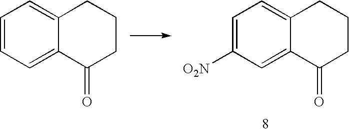

Concentrated sulfuric acid (60 ml) was cooled to 0° C. in an ice bath. α-Tetralone (8 g, 54.7 mmol) was added with stirring, then potassium nitrate (6 g, 59.3 mmol, 1.08 equiv.) dissolved in concentrated sulfuric acid (18 ml) was added dropwise via a dropping funnel, making sure that the temperature of the solution did not rise above 15° C. After addition, the solution was stirred for 1 h and then poured into crushed ice. The precipitate was filtered and washed with distilled water and then left to dry. Recrystallisation from a ethanol/water (1:1) yielded 8 as a slightly yellow solid (8.5 g, 81%), m.p. 104-106° C.; I.R. (film)/cm⁻¹ 1675, 1500, 1340; ¹H NMR (400 MHz, CDCl₃) 2.18-2.25 (2H, m, CH₂), 2.75 (2H, t, J 6.8, CH₂), 3.10 (2H, t, J 6.1, CH₂), 7.45 (1H, d, J 8.4, ArH), 8.30 (1H, dd, J 2.4, 8.4, ArH), 8.86 (1H, d, J 2.4, ArH).

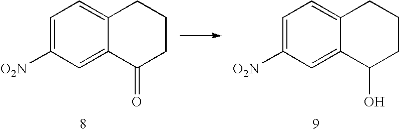

Sodium borohydride (6.95 g, 183.9 mmol) was added to a solution of 3,4-dihydro-7-nitro-1(2H)-naphthalenone (8 g, 41.8 mmol) in ethanol (240 ml) at 0° C. After the vigorous reaction subsided, the cooling bath was removed and the solution was then stirred for a further 10 min. Hydrochloric acid (2M) was then added and the crude reaction mixture was then extracted with ethyl acetate. Column chromatography on silica gel eluting with petroleum ether:ethyl acetate 4:1 gave alcohol 9 as a pale green solid (7.9 g, 98%), m.p. 107-109° C.; I.R. (film)/cm⁻¹ 3500; ¹H NMR (400 MHz, CDCl₃) 1.73-2.26 (4H, m, 2×CH₂), 2.29 (1H, bs, OH), 2.78-2.91 (2H, m, CH₂), 4.78 (1H, m, CH), 7.17-7.26 (1H, d, J 8.4, ArH), 7.92-8.12 (1H, dd, J 2.4, 8.4, ArH), 8.30 (1H, d, J 2.4, ArH).

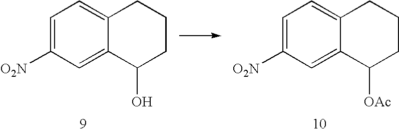

An excess of acetic anhydride (2.4 ml) was added to a solution of 7-nitro-1,2,3,4-tetrahydronaphthalen-1-ol 9 (0.45 g, 2.33 mmol.) in pyridine (3.2 ml). The reaction mixture was stirred for 16 h at room temperature and then worked up to give the crude acetate 10. Column chromatography on silica gel eluting with petroleum ether:ethyl acetate 5:1 gave pure acetate 10 as a slightly yellow oil (0.46 g, 84%); ¹H NMR (400 MHz, CDCl₃) 1.84-2.10 (4H, m, 2×CH₂), 2.13 (3H, s, CH₃), 2.80-3.00 (2H, m, CH₂), 6.00 (1H, t, J 4.8, CH), 7.28 (1H, d, J 8.4, ArH), 8.07 (1H, dd, J 2.4, 8.4, ArH), 8.16 (1H, d, J 2.4, ArH).

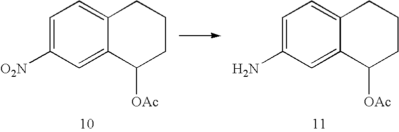

Copper (II) acetylacetonate (94 mg, 0.36 mmol) was dissolved in ethanol (70 ml) and sodium borohydride (68 mg, 1.79 mmol) was added under nitrogen. The reaction mixture was further stirred for 1 h at which time a black precipitate was formed. At this time ethanol (74 ml) and acetate 10 (0.42 g, 1.79 mmol) were added followed by sodium borohydride (135 mg, 3.58 mmol). The reaction was stirred for a further 2 h. Then water was added and the solvent was removed in vacuo. After this, the mixture was diluted with diethyl ether and washed with brine, the ether layer was dried over anhydrous sodium sulfate, filtered and the solvent was removed in vacuo. Column chromatography on silica gel eluting with petroleum ether:ethyl acetate 2:1 gave amine 11 as a yellow oil (0.36 g, 98%); ¹H NMR (400 MHz, CDCl₃) 1.74-1.97 (4H, m, 2×CH₂), 2.09 (3H, s, CH₃), 2.59-2.78 (2H, m, CH₂), 3.57 (2H, bs, NH₂), 5.91 (1H, t, J 4.4, CH), 6.60 (2H, m, ArH), 6.93 (1H, d, J 7.7, ArH).

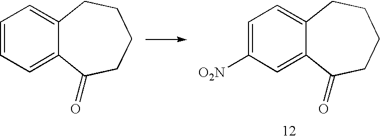

Concentrated sulfuric acid (55 ml) was cooled to 0° C. in an ice bath. 1-Benzosuberone (8 g, 49.9 mmol) was added with stirring, then potassium nitrate (5.55 g, 54.9 mmol) dissolved in concentrated sulfuric acid (15 ml) was added dropwise via a dropping funnel, making sure that the temperature of the solution did not rise above 15° C. After addition, the solution was stirred for 1 h and then poured into crushed ice. The precipitate was filtered and washed with distilled water and then left to dry. Recrystallisation from ethanol/water 1:1 yielded nitro derivative 12 as a pale yellow solid (7.98 g, 78%), m.p. 91-93° C. (lit. m.p. 92-93° C.); ¹³C NMR (100.61 MHz, CDCl₃) 25.1 (t), 31.5 (t), 32.1 (t), 38.9 (t), 123.6 (d), 127.9 (d), 129.1 (d), 138.2 (s), 145.6 (s), 146.1 (s), 197.6 (s).

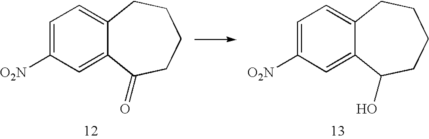

Sodium borohydride (4.9 g, 130.2 mmol) was added to a solution of nitroderivative 12 (6.1 g, 29.6 mmol) in ethanol (160 ml) at 0° C. After the vigorous reaction subsided, the cooling bath was removed and the solution was then stirred for a further 10 min. Hydrochloric acid (2M) was then added and the crude reaction mixture was then extracted with ethyl acetate. Column chromatography on silica gel eluting with petroleum ether:ethyl acetate 5:1 gave alcohol 13 as a pale yellow solid (6.0 g, 98%), m.p. 115-117° C.; ¹H NMR (400 MHz, CDCl₃) 1.61-1.93 (3H, m, CH₂), 2.05-2.13 (3H, m, CH₂), 2.78 (1H, m, CH₂), 3.04 (1H, m, CH₂), 5.02 (1H, m, CH), 7.25 (1H, d, J 8.8, ArH), 8.03 (1H, dd, J 2.3, 8.8, ArH), 8.42 (1H, d, J 2.3, ArH).

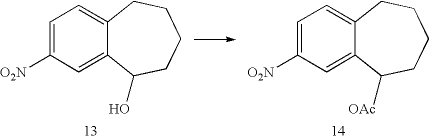

An excess of acetic anhydride (9 ml) was added to a solution of alcohol 13 (1.5 g, 7.2 mmol) in pyridine (15 ml). The reaction mixture was stirred for 16 h at room temperature and then extracted, evaporated filtered and dried to give the crude acetate. Column chromatography on silica gel eluting with petroleum ether:ethyl acetate 6:1 gave pure the acetate 14 as a colourless oil (1.69 g, 94%); ¹H NMR (400 MHz, CDCl₃) 1.62-1.80 (2H, m, CH₂), 1.89-2.09 (4H, m, 2×CH₂), 2.18 (3H, s, CH₃), 2.73 (1H, m, CH₂), 2.97 (1H, m, CH₂), 5.96 (1H, t, J 7.7, CH), 7.28 (1H, d, J 8.8, ArH), 8.09 (1H, dd, J 2.3, 8.8, ArH), 8.48 (1H, d, J 2.3, ArH).

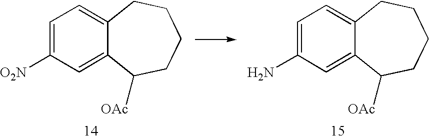

Copper (II)acetylacetonate (440 mg, 1.68 mmol) was dissolved in ethanol (300 ml) and sodium borohydride (319.3 mg, 8.4 mmol) was added under nitrogen. The reaction mixture was further stirred for 1 h at which time a black precipitate had formed. Ethanol (350 ml) and acetate ester 14 (2.1 g, 8.4 mmol) were added, followed by sodium borohydride (638.7 mg, 16.8 mmol). The reaction was stirred for a further 2 h. Then water was added and the solvent was removed in vacuo. After this, the residue was dissolved in diethyl ether and washed with brine, dried over anhydrous sodium sulfate, filtered and the solvent was removed in vacuo. Column chromatography on silica gel eluting with petroleum ether:ethyl acetate 2:1 gave amine 15 as a yellow solid (1.77 g, 96%), m.p. 84-86° C.; ¹H NMR (400 MHz, CDCl₃) 1.52-1.65 (1H, m, CH₂), 1.68-1.79 (1H, m, CH₂), 1.80-1.99 (4H, m, 2×CH₂), 2.15 (3H, s, CH₃), 2.62-2.69 (1H, m, CH₂), 2.82-2.88 (1H, m, CH₂), 5.84 (1H, t, J 7.7, CH), 6.49 (1H, dd, J 2.5, 7.9, ArH), 6.68 (1H, d, J 2.5, ArH), 6.90 (1H, d, J 7.9, ArH).

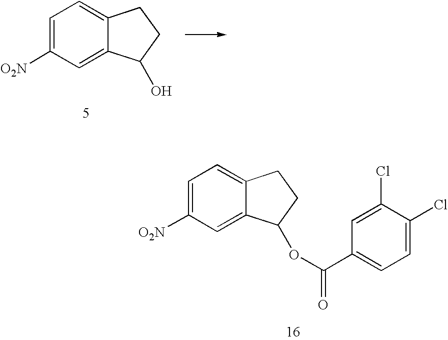

3,4-Dichlorobenzoyl chloride (3.1 g, 14.8 mmol) was added to a solution of crude 5 (2.4 g, 13.4 mmol) in pyridine (10 mL) under nitrogen at 0° C., and the mixture was stirred at room temperature overnight before being poured into water, and then extracted with dichloromethane. The organic phase was washed with hydrochloric acid (2M), dried over magnesium sulfate, filtered and concentrated. Purification by flash chromatography (petroleum ether/ethyl acetate, 85/15) gave compound 16 (3.78 g, 80%) as a white solid. ¹H NMR (CDCl₃, 400 MHz): 2.31-2.39 (1H, m, CH₂), 2.70-2.79 (1H, m, CH₂), 3.02-3.10 (1H, m, CH₂), 3.24-3.33 (1H, m, CH₂), 6.45-6.48 (1H, m, CHO), 7.47 (1H, d, J 8.3, ArH), 7.53 (1H, d, J 8.3, ArH), 7.87 (1H, dd, J, 8.3, 2.0, ArH), 8.10 (1H, d, J, 2.0, ArH), 8.22 (1H, dd, J, 8.3, 2.0, ArH), 8.33 (1H, d, J, 2.0, ArH).

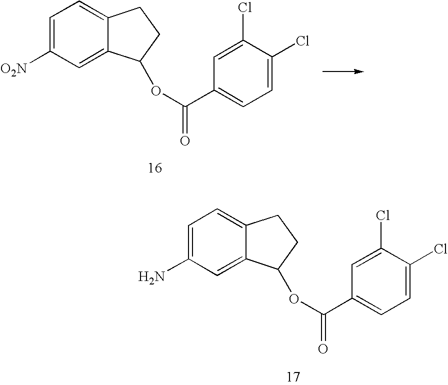

A solution of 16 (2.0 g, 5.68 mmol) in ethyl acetate (10 mL) was subjected to hydrogenation at atmospheric pressure with Pd/C as catalyst. The reaction was followed carefully by TLC and was stopped when most of the starting material was consumed. The mixture was filtered through kieselguhr and was concentrated. Purification by flash chromatography (petroleum ether/ethyl acetate, 70/30) gave amine 17 (823 mg, 45%) as a pale brown solid. ¹H NMR (CDCl₃, 400 MHz): 2.17-2.25 (1H, m, CH₂), 2.55-2.64 (1H, m, CH₂), 2.81-2.92 (1H, m, CH₂), 2.05-3.12 (1H, m, CH₂), 3.66 (2H, broad, NH₂), 6.34-6.50 (1H, m, CHO), 6.69 (1H, dd, J 8.0, 2.0, ArH), 6.81 (1H, d, J 2.0, ArH), 7.10 (1H, d, J 8.0, ArH), 7.50 (1H, d, J, 8.4, ArH), 7.87 (1H, dd, J, 8.4, 1.9, ArH), 8.10 (1H, d, J, 1.9, ArH).

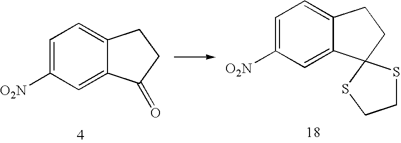

To a solution of 4 (2.40 g, 13.56 mmol) in dry DCM (20 mL) under nitrogen atmosphere was added 1,2-ethanedithiol (1.915 g, 20-34 mmol, 1.71 mL) and BF₃.OEt₂ (1.92 g, 13.56 mmol, 1.67 mL). The mixture was stirred at room temperature for 2 hours and was diluted with DCM (50 mL) washed with aqueous NaOH (10%), dried over MgSO₄, filtered and concentrated to give 18 as a yellow oil (3.26 g, 95%), which was used in next reaction without purification. ¹H NMR (CDCl₃, 400 MHz): 2.76 (2H, t, J 6.75, CH₂), 3.05 (2H, t, J 6.75, CH₂), 3.44-3.51 (2H, m, CH₂), 3.55-3.62 (2H, m, CH₂), 7.31 (1H, d, J 8.28, ArH), 8.08 (1H, dd, J 8.28, 2.19, ArH), 8.35 (1H, d, J 2.19, ArH).

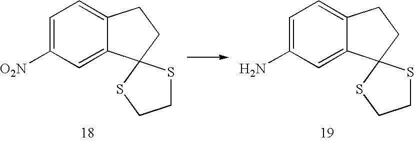

A solution of 18 (500 mg, 1.976 mmol) and SnCl₂.2H₂O (2.23 g, 9.88 mmol) in MeOH (15 mL) was refluxed for 4 hours and then stirred at room temperature overnight. The mixture was quenched by adding saturated aqueous NaHCO₃ (30 mL) carefully, extracted with ethyl acetate (50 mL), the organic phase was dried over MgSO₄, filtered and concentrated. Purification by flash chromatography (petroleum ether/EtOAc 75/25) gave compound 19 as a yellow oil (315 mg, 71%). ¹H NMR (CDCl₃, 400 MHz): 2.64 (2H, t, J 6.55, CH₂), 2.84 (2H, t, J 6.55, CH₂), 3.38-3.41 (2H, m, CH₂), 3.46-3.55 (2H, m, CH₂), 3.65 (2H, broad, NH₂), 6.54 (1H, dd, J 7.97, 2.07, ArH), 6.89 (1H, d, J 2.07, ArH), 6.95 (1H, d, J 7.97, ArH).

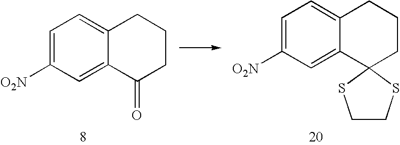

To a solution of 8 (1.0 g, 5.235 mmol) in dry DCM (10 mL) was added 1,2-ethanedithiol (740 mg, 7.853 mmol, 0.66 mL) and BF₃.OEt₂ (1.113 g, 7.853 mmol, 0.96 mL) under nitrogen atmosphere. The mixture was stirred at room temperature overnight, diluted with DCM (50 mL), washed with 2N NaOH, dried over MgSO₄, filtered and concentrated. Compound 20 was obtained as slightly yellow solid (1.4 g, 100%) and was used without purification in next reaction. ¹H NMR (CDCl₃, 400 MHz): 2.02-2.08 (2H, m, CH₂), 2.40-2.43 (2H, m, CH₂), 2.87 (2H, t, J 6.38, CH₂), 3.47-3.54 (2H, m, CH₂), 3.62-3.70 (2H, m, CH₂), 7.14 (1H, d, J 8.49, ArH), 7.93 (1H, dd, J 8.49, 2.40, ArH), 8.80 (1H, d, J 2.40, ArH).

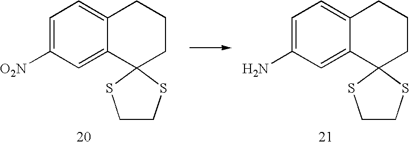

A suspension of 20 (1.4 g, 5.235 mmol) and SnCl₂.2H₂O in MeOH (30 mL) was refluxed under nitrogen atmosphere for 4 hours. The mixture was cooled to room temperature and poured into 100 mL of saturated NaHCO₃, the mixture was extracted with EtOAc, dried over MgSO₄, filtered and concentrated. Purification by flash chromatography afforded compound 21 as a slightly yellow oil (1.0 g, 80%). ¹H NMR (CDCl₃, 400 MHz): 1.62-2.00 (2H, m, CH₂), 2.30-2.39 (2H, m, CH₂), 2.70 (2H, t, J 6.41, CH₂), 3.30-3.34 (2H, m, CH₂), 3.38-3.61 (4H, m, CH₂+NH₂), 6.51 (1H, dd, J 8.12, 2.28, ArH), 6.80 (1H, d, J 2.48, ArH), 7.29 (1H, d, J 8.12, ArH).

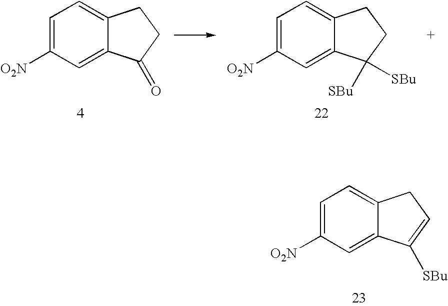

To a solution of 4 (1.0 g, 5.65 mmol) in chloroform (10 mL) under nitrogen atmosphere at room temperature was added n-butanethiol (1.27 g, 14.124 mmol, 1.513 mL) and chlorotrimethylsilane (1.534 g, 14.124 mmol, 1.805 mL). The mixture was stirred at room temperature overnight and was diluted with DCM (20 mL), washed with 2N NaOH, dried over MgSO₄, filtered and concentrated. Purification by column (petro-leum ether/ether 95/5) gave compound 22 (slightly yellow oil, 1.27 g, 77%) and 23 (yellow solid, 290 mg, 20%). ¹HNMR for 22: ¹H NMR (CDCl₃, 400 MHz): 0.89 (6H, t, J 7.28, CH₃). 1.32-1.43 (4H, m, CH₂), 1.45-1.61 (4H, m, CH₂), 2.46-2.53 (2H, m, CH₂), 2.59-2.67 (4H, m, CH₂), 3.10 (2H, t, J 6.92, CH₂), 7.38 (1H, d, J 8.06, ArH), 8.11-8.14 (2H, m, ArH). ¹HNMR for 23: ¹H NMR (CDCl₃, 400 MHz): 0.97 (3H, t, J 7.35, CH₃). 1.46-1.57 (2H, m, CH₂), 1.71-1.78 (2H, m, CH₂), 3.00 (2H, t, J 7.33, CH₂), 3.56 (2H, d, J 2.29, CH₂), 6.35 (1H, t, J 2.29, CH), 7.56 (1H, d, J 8.14, ArH), 8.14 (1H, dd, J 8.14, 2.11, ArH), 8.22 (1H, d, J 2.11, ArH).

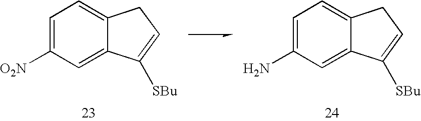

A solution of 23 (40 mg, 0.16 mmol) in EtOAc (10 mL) was subjected to hydrogenation at atmospheric pressure with Pd/C as catalyst overnight. After filtration, the solvent was removed to give a residue (20 mg), that was used in the coupling reaction without further purification.

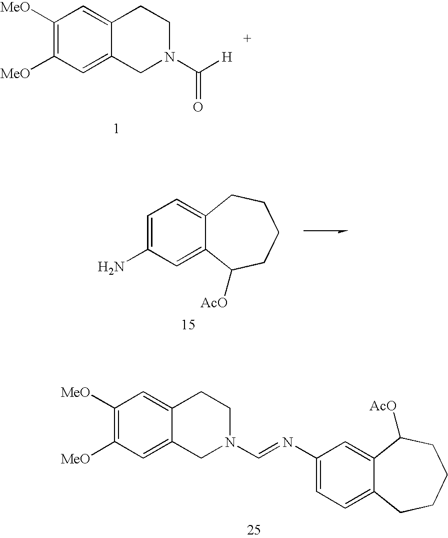

25 m.p. 160° C. [decomp.]; (Found: MH⁺ 423.2276, C₂₅H₃₀N₂O₄ requires MH 423.2284); m/z (EI) 423 ([M+H]⁺, 5%), 362 (65), 192 (48), 177 (50), 115 (60), 44 (85), 43 (100).

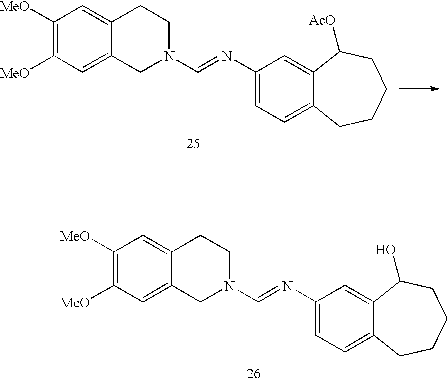

26 ¹H NMR (CDCl₃, 400 MHz): 1.35-1.43 (1H, m, CH₂), 1.68-1.80 (3H, m, CH₂), 1.90-2.05 (2H, m, CH₂), 2.24 (1H, broad OH), 2.61-2.86 (4H, m, CH₂), 3.68 (2H, broad, NCH₂), 3.85 (3H, s, CH₃), 3.86 (3H, s, CH₃), 4.63 (2H, broad, NCH₂), 4.85-4.88 (1H, m, OCH), 6.63 (1H, s, ArH), 6.64 (1H, s, ArH), 6.76 (1H, dd, J 7.81, 1.83 ArH), 6.97 (1H, d, J 7.81, ArH), 7.07 (1H, d, J 1.83, ArH), 7.70 (1H, s, N═CH). MS: C₂₃H₂₈N₂O₃, M+H, calculated 381.2178, found 381.2178

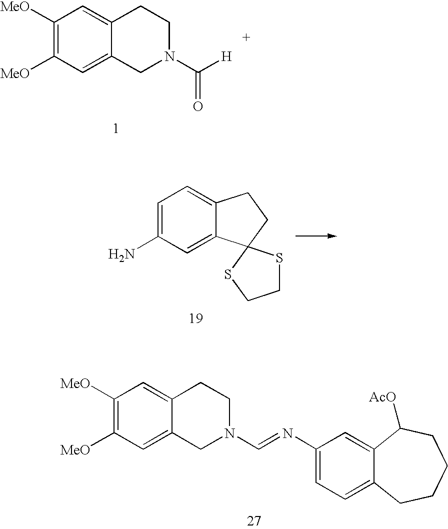

27 ¹H NMR (CDCl₃, 400 MHz): 2.69 (2H, t, J 6.58, CH₂), 2.86 (2H, t, J 5.69, CH₂), 2.92 (2H, t, J 6.58, CH₂), 3.40-3.46 (2H, m, CH₂), 3.50-3.56 (2H, m, CH₂), 3.68 (2H, broad, NCH₂), 3.86 (3H, s, CH₃), 3.87 (3H, s, CH₃), 4.66 (2H, broad, NCH₂), 6.64 (1H, s, ArH), 6.65 (1H, s, ArH), 6.87 (1H, dd, J 7.95, 1.89 ArH), 7.08 (1H, d, J 7.95, ArH), 7.17 (1H, d, J 1.89, ArH), 7.71 (1H, s, N═CH). MS: C₂₃H₂₆N₂O₂S₂, M+H, calculated 427.1514, found 427.1514

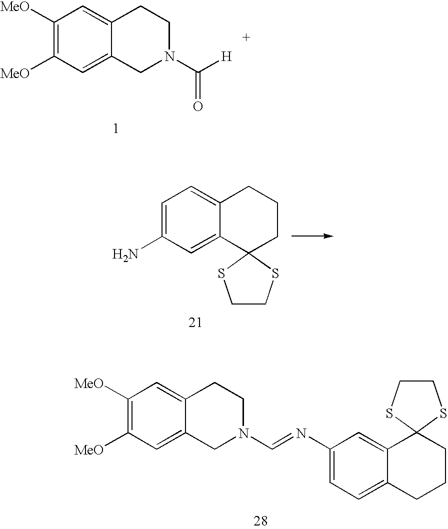

28 ¹H NMR (CDCl₃, 400 MHz): 2.69 (2H, t, J 6.58, CH₂), 1.97-2.02 (2H, m, CH₂), 2.37-2.40 (2H, m, CH₂), 2.76 (2H, t, J 5.91, CH₂), 2.85 (2H, t, J 5.66, CH₂), 3.40-3.48 (2H, m, CH₂), 3.68 (2H, broad, NCH₂), 3.85 (3H, s, CH₃), 3.87 (3H, s, CH₃), 4.65 (2H, broad, NCH₂), 6.63 (1H, s, ArH), 6.64 (1H, s, ArH), 6.78 (1H, dd, J 8.09, 2.12 ArH), 6.90 (1H, d, J 8.09, ArH), 7.55 (1H, d, J 2.12, ArH), 7.67 (1H, s, N═CH). MS: C₂₄H₂₈N₂O₂S₂, M+H, calculated 441.1670, found 441.1662.

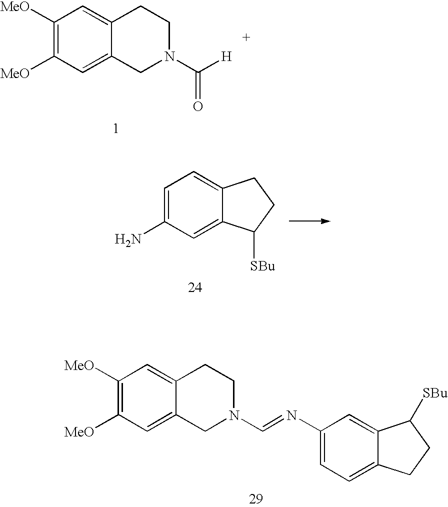

29 ¹H NMR (CDCl₃, 400 MHz): 0.92 (3H, t, J 7.33, CH₃), 1.38-1.46 (2H, m, CH₂), 1.56-1.64 (2H, m, CH₂), 2.11-2.19 (1H, m, CH₂), 2.49-2.60 (3H, m, CH₂), 2.78-2.88 (3H, m, CH₂), 2.98-3.05 (1H, m, CH₂), 3.68 (2H, broad, NCH₂), 3.86 (3H, s, CH₃), 3.87 (3H, s, CH₃), 4.29 (1H, dd, J 7.35, 5.35, SCH), 4.65 (2H, broad, NCH₂), 6.64 (1H, s, ArH), 6.65 (1H, s, ArH), 6.84 (1H, dd, J 7.92, 1.75 ArH), 6.97 (1H, d, J 1.75, ArH), 7.11 (1H, d, J 7.92, ArH), 7.69 (1H, s, N═CH).

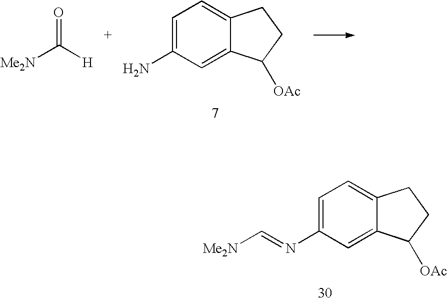

30 ¹H NMR (CDCl₃, 400 MHz): 2.03-2.14 (4H, m, Ac+CH₂), 2.44-2.53 (1H, m, CH₂), 2.76-2.87 (1H, m, CH₂), 2.94-3.07 (7H, m, NMe₂+CH₂), 6.12-6.17 (1H, m, CHO), 6.90 (1H, dd, J, 7.9, 1.7 Hz, ArH), 6.97 (1H, d, J, 1.7 Hz, ArH). 7.13 (1H, 7.9 Hz, ArH), 7.51 (1H, s, CH═N).

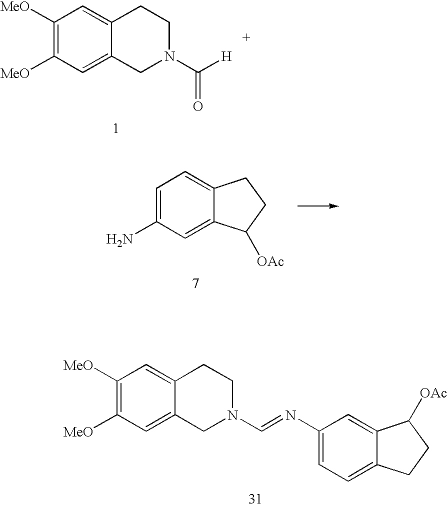

31 ¹H NMR (CDCl₃, 400 MHz): 2.01-2.13 (4H, m, Ac+CH₂), 2.46-2.55 (1H, m, CH₂), 2.79-2.88 (3H, m, CH₂), 3.02-3.10 (1H, m, CH₂), 3.67 (2H, broad, CH₂), 3.86 (3H, s), 3.87 (3H, s), 4.66 (2H, broad, CH₂), 6.17-6.20 (1H, m, CHO), 6.64 (1H, s, ArH), 6.65 (1H, m, ArH), 6.95 (1H, d, J, 8.0 Hz, ArH), 7.01 (1H, s, ArH), 7.16 (1H, d, J, 8.0 Hz, ArH), 7.69 (1H, s, CH═N).

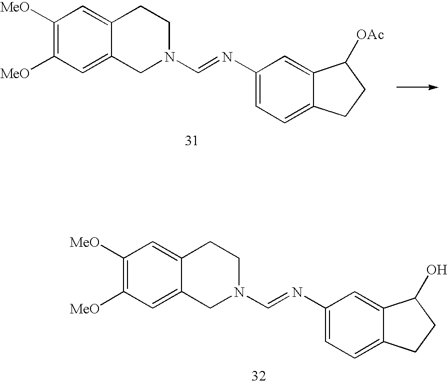

32 ¹H NMR (CDCl₃, 400 MHz): 1.90-1.98 (1H, m), 2.17 (1H, broad), 2.44-2.52 (1H, m), 2.72-2.77 (1H, m), 2.84-2.87 (2H, m), 2.95-3.02 (1H, m), 3.70 (2H, broad), 3.85 (3H, s), 3.87 (3H, s), 4.65 (2H, broad), 5.19 (1H, t, J, 6.1, CHO), 6.63 (1H, s, ArH), 6.64 (1H, s, ArH), 6.90 (1H, d, J, 7.9, ArH), 7.02 (1H, s, ArH), 7.12 (1H, d, J, 7.9, ArH), 7.69 (1H, CH═N).

MS: C₂₁H₂₄N₂O₃, M+H, calculated 353.1865, found 353.1859

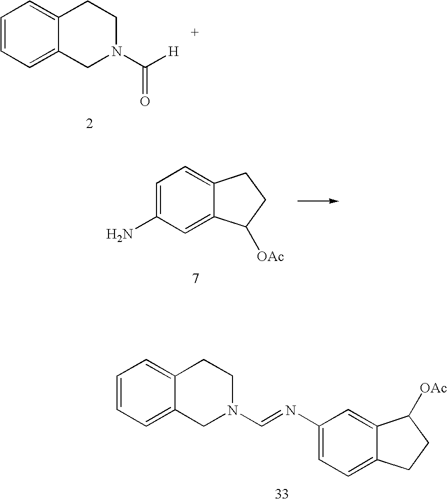

33 ¹H NMR CDCl₃, 400 MHz): 2.15-2.23 (4H, m, Ac+CH₂), 2.55-2.64 (1H, m, CH₂), 2.88-2.96 (1H, m, CH₂), 3.01-3.02 (2H, m, CH₂), 3.10-3.19 (1H, m, CH₂), 3.79 (2H, broad, CH₂), 4.82 (2H, broad, CH₂), 6.26-6.29 (1H, m, CHO), 7.05 (1H, d, J, 7.9, ArH), 7.12 (1H, s, ArH), 7.25-7.35 (5H, m, ArH), 7.79 (1H, s, CH═N).

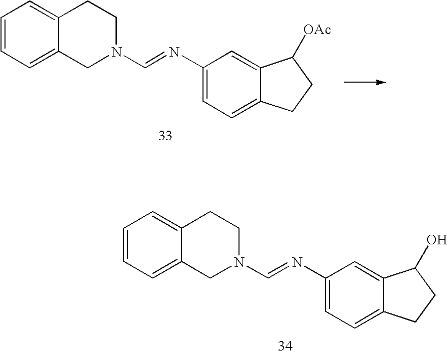

34 ¹H NMR (CDCl₃, 400 MHz): 1.91-2.00 (1H, m), 2.44-2.52 (1H, m), 2.72-2.80 (2H, m), 2.93-3.03 (3H, m), 3.69 (2H, broad), 4.70 (2H, broad), 5.20 (1H, t, J, 6.2, CHO), 6.92 (1H, d, J, 7.9 Hz, ArH), 7.05 (1H, m, ArH), 7.13-7.28 (5H, m, ArH), 7.70 (1H, s, CH═N).

MS: C₁₉H₂₀N₂O, M+H, calculated 293.1654, found 293.1651

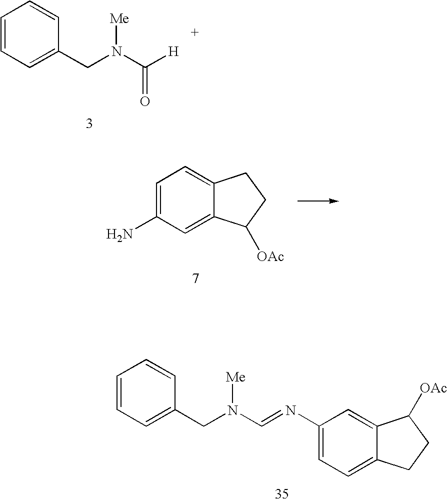

35 ¹H NMR (CDCl₃, 400 MHz): 2.10-2.22 (4H, m, Ac+CH₂), 2.55-2.64 (1H, m, CH₂)₁ 2.88-2.95 (1H, m, CH₂), 3.03 (3H, s, NMe), 3.11-3.18 (1H, m, CH₂), 4.50 (2H, broad, CH₂), 6.26-6.29 (1H, m, CHO), 7.06 (1H, d, J, 7.9, ArH), 7.14 (1H, s, ArH), 7.26 (1H, d, J, 7.9, ArH), 7.34-7.46 (5H, m, ArH), 7.83 (1H, broad, CH═N). MS: C₂₀H₂₂N₂O₂, M+H, calculated 323.1759, found 323.1765.

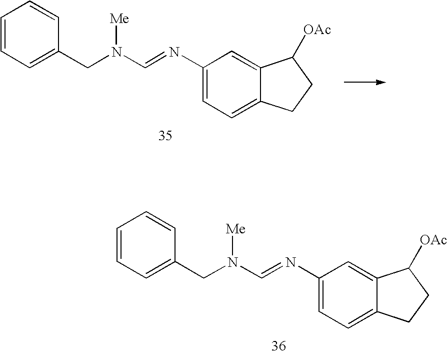

36 ¹H NMR (CDCl₃, 400 MHz): 1.90-1.99 (1H, m, CH₂), 2.30 (1H, broad, OH), 2.44-2.53 (1H, m, CH₂), 2.72-2.80 (1H, m, CH₂), 2.95-3.11 (4H, m, NMe+CH₂), 4.50 (2H, broad, CH₂), 5.13-5.25 (1H, m, CHO), 6.92-6.94 (1H, m, ArH), 7.05 (1H, s, ArH), 7.13-7.16 (1H, m, ArH), 7.27-7.39 (5H, m, ArH), 7.76 (1H, broad, CH═N). MS: C₁₈H₂₀N₂O, M+H, calculated 281.1654, found 281.1648.

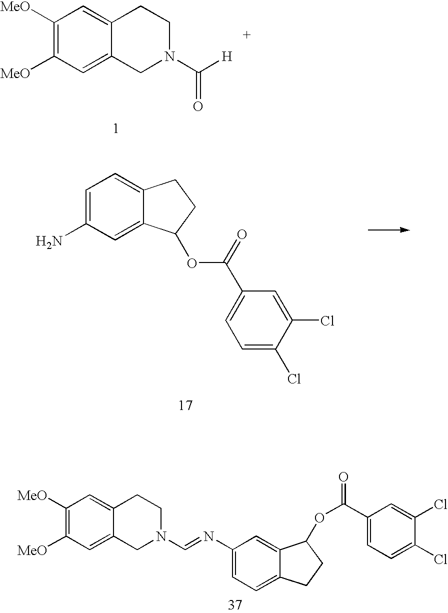

37 ¹H NMR (CDCl₃, 400 MHz): 2.19-2.28 (1H, m, CH₂), 2.58-2.67 (1H, m, CH₂), 2.84-2.94 (3H, m, CH₂), 3.10-3.18 (1H, m, CH₂), 3.64 (2H, broad, CH₂), 3.85 (3H, s, OMe), 3.86 (3H, s, OMe), 4.68 (2H, broad, CH₂), 6.40-6.43 (1H, m, CHO), 6.62 (2H, s, ArH), 6.98 (1H, dd, J, 7.7, 1.6 Hz, ArH), 7.08 (1H, d, J, 1.6, ArH), 7.19 (1H, d, J, 7.7, ArH), 7.48 (1H, d, J, 8.3, ArH), 7.69 (1H, s, CH═N), 7.86 (1H, dd, J, 8.3, 1.9 Hz, ArH), 8.10 (1H, d, J, 1.9, ArH).

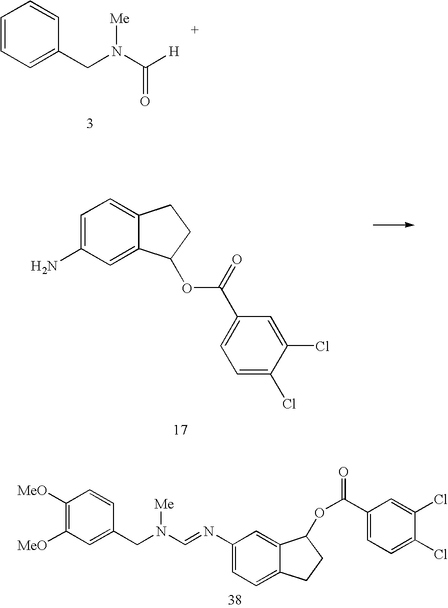

38 ¹H NMR (CDCl₃, 400 MHz): 2.27-2.34 (1H, m, CH₂), 2.65-2.74 (1H, m, CH₂), 2.93-3.00 (4H, m, NMe+CH₂), 3.18-3.25 (1H, m, CH₂), 4.50 (2H, broad, CH₂), 6.48-6.51 (1H, m, CHO), 7.06 (1H, dd, J, 8.0, 1.8, ArH), 7.17 (1H, d, J, 1.8, ArH), 7.27-7.43 (6H, m, ArH), 7.54-7.56 (1H, m, ArH), 7.81 (1H, broad, CH═N), 7.93 (1H, dd, J, 8.4, 1.9, ArH), 8.18 (1H, d, J, 1.9, ArH).

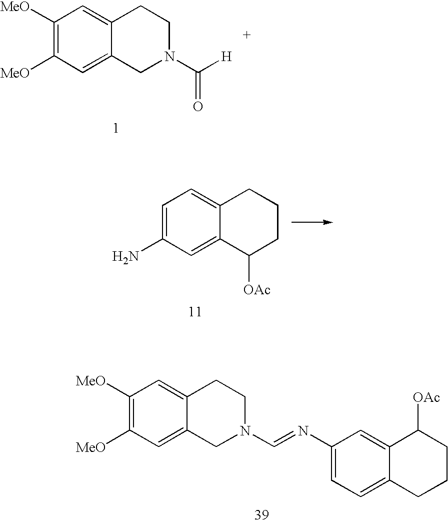

39 ¹H NMR (CDCl₃, 400 MHz): 1.78-1.95 (4H, m, CH₂), 2.06 (3H, s, Ac), 2.65-2.85 (4H, m, CH₂), 3.61 (2H, broad, CH₂), 3.83-3.84 (6H, m, OMe), 4.66 (2H, broad, CH₂), 5.96 (1H, m, CHO), 6.61 (2H, s, ArH), 6.87-6.88 (2H, m, ArH), 7.00-7.03 (1H, m, ArH), 7.65 (1H, s, CH═N).

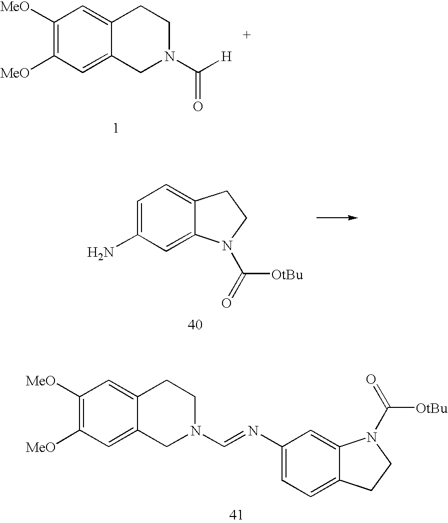

41 ¹HNMR (DMSO, 400 MHz): 1.51 (9H, s, C(CH₃)₃), 2.92-3.08 (4H, m, ArCH₂), 3.75-3.76 (6H, m, OMe), 3.93-3.98 (4H, m, NCH₂), 4.86-4.90 (2H, m, ArCH₂N), 6.69 (1H, s, ArH), 6.81-6.89 (2H, m, ArH), 7.01-7.10 (1H, m, ArH), 7.25-7.28 (1H, m, ArH), 8.32 (1H, s, NCH═N), 8.82 (1H, broad, NH).

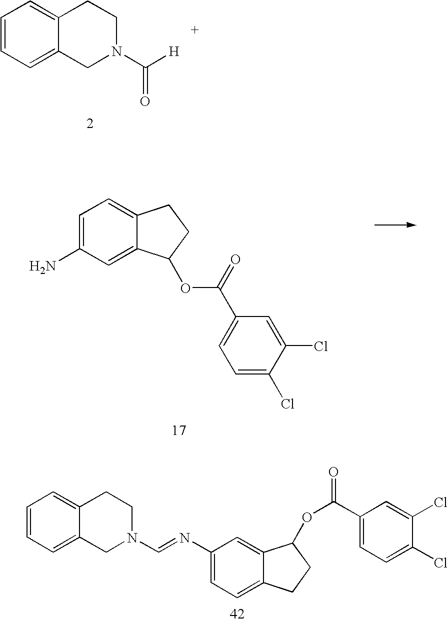

42 ¹HNMR (CDCl₃, 400 MHz): 2.26-2.39 (2H, m, CH₂), 2.65-2.74 (1H, m, CH₂), 2.94-3.02 (2H, m, CH₂), 3.17-3.25 (1H, m, CH₂), 4.70 (2H, broad, CH₂N), 4.82 (2H, broad, ArCH₂N), 6.44-6.50 (1H, m, OCH), 7.04-7.05 (1H, m, ArH), 7.06-7.07 (1H, m, ArH), 7.14-7.38 (5H, m, ArH), 7.55-7.60 (1H, m, ArH), 7.76 (1H, s, NCH═N), 7.90-7.94 (1H, m, ArH), 8.15-8.17 (1H, m, ArH).

44 ¹HNMR (CDCl₃, 400 MHz): 1.90-1.98 (1H, m, CH₂),

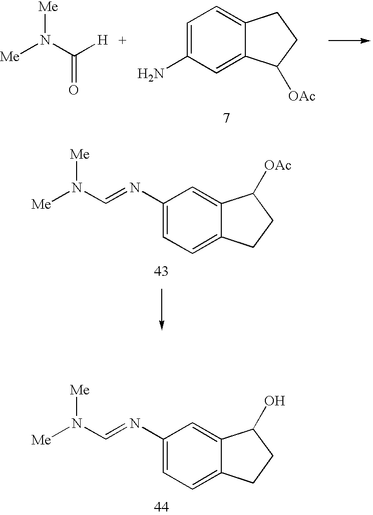

2.41-2.52 (1H, m, CH₂), 2.67-2.79 (1H, m, CH₂), 2.95-3.01 (7H, m, NCH₃+CH₂), 5.19 (1H, t, J 6.05 Hz), 6.86-6.88 (1H, m, ArH), 6.99-7.00 (1H, m, ArH), 7.11-7.13 (1H, m, ArH), 7.51-7.52 (1H, s, NCH═N). ¹³CNMR (CDCl₃, 100 MHz): 29.37 (CH₂), 36.34 (CH₂), 76.72 (CH), 116.44 (CH), 122.10 (CH), 125.39 (CH), 137.41 (C), 146.24 (C), 151.26 (C), 153.71 (CH).

### REFERENCES

Each of the following references is specifically incorporated herein by reference. In addition, one skilled in the art can rely on the contents of these references to make and use embodiments of this invention.
Pouzet, B., Didriksen, M., & Arnt J. (2002) Effects of the 5-HT₇ receptor antagonist SB-258741 in animal models for schizophrenia. Pharmacology Biochemistry And Behavior 71, 655-665
Shannon, H. E., Bymaster, F. P., Rasmussen, K., DeLapp, N. W., Calligaro, D. O., Perry, K. W., Mitch, C. H., Ward, J. S., Fink-Jensen, A., Olesen, P., Rasmussen, T., Sheardown, M., Swedberg, M., Jeppesen, L., & Sauerberg, P. (1999a) Behavioral and electrophysiological effects of the antipsychotic-like muscarinic agonist PTAC. Life Sciences 64, 67-71
Shannon, H. E., Rasmussen, K., Bymaster, F. P., Hart, J. C., Peters, S. C., Swedberg, M., Jeppesen, L., Sheardown, M., Sauerberg, P. & Fink-Jensen, A., (1999b) Xanomeline, an M₁/M₄ preferring muscarinic cholinergic receptor agonist, produces antipsychotic-like activity in rats and mice. Life Sciences 64, 67-71
Bymaster, F. P., Shannon, H. E., Rasmussen, K., Delapp, N. W., Mitc,h C. H., Ward J. S., Calligaro, D. O., Ludvigsen, T. S., Sheardown, M. J., Olesen, P. H., Swedberg M. D. B., Sauerberg P., Fink-Jensen, A. (1998) Unexpected antipsychotic-like activity with the muscarinic receptor ligand (5R,6R)6-(3-propylthio-1,2,5-thiadiazol-4-yl)-1-azabicyclo[3.2.1]octane. European Journal Of Pharmacology 356, 109-119
Allen, R. M. & Young, S. J. Phencyclidine-induced psychosis. Am. J. Psychiatry 135, 1081-1084 (1978).
Andreasen, N. C., Rezai, K., Alliger, R., Swayze, V. W. d., Flaum, M., Kirchner, P., Cohen, G. & O'Leary, D. S. (1992). Hypofrontality in neuroleptic-naive patients and in patients with chronic schizophrenia. Assessment with xenon 133 single-photon emission computed tomography and the Tower of London. Archives of General Psychiatry, 49, 943-58.
Braff D., Stone C., Callaway E., Geyer M., Glick I., Bali L. (1978) Prestimulus effects on human startle reflex in normals and schizophrenics. Psychophysiology 15, 339-343.
Buchsbaum, M. S., Potkin, S. G., Marshall, J. F., Lottenberg, S., Teng, C., Heh, C. W., Tafalla, R., Reynolds, C., Abel, L., Plon, L. & al, e. (1992). Effects of clozapine and thiothixene on glucose metabolic rate in schizophrenia. neuropsychopharmacology, 6, 155-63.
Buchsbaum, M. S., Someya, T., Teng, C. Y., Abel, L., Chin, S., Najafi, A., Haier, R. J., Wu, J. and Bunney, W. E. Jr. (1996). PET and MRI of the thalamus in never-mediacated patients with schizophrenia. Am. J. Psychiatry, 153, 191-199.
Cochran, S., McKerchar, C. M., Steward, L., Pratt, J. A. & Morris, B. J. (2002) Neuropsychopharmacology 28, 265-275.
Cochran, S., McKerchar, C. M., Steward, L., Pratt, J. A. & Morris, B. J. (2003) Neuropsychopharmacology (in press).
Crane, A. M. and Porrino, L. J. (1989). Adaptation of the quantitative 2-[14-C]-deoxyglucose method for use in freely moving rats. Brain Research., 499, 87-92.
Crook, J. M., Tomaskovic-Crook, E., Copolov, D. L. & Dean, B. (2001) Low muscarinic receptor binding in preforntal cortex from subjects with schizophrenia. Am. J. Psychiatry 158, 918-925.
Eglen, (2001) Therapeutic opportunities from muscarinic receptor research. Trends in Pharmacological Sciences, 22:409-414
Goldberg, T., Greenberg, R., Griffen, S., Gold, J., Pickar, D., Kleinman, J. and Weinberger, D. (1993). The impact of clozapine on cognitive impairment and psychiatric symptomatology in patients with schizophrenia. Br. J. Psychiatry 162, 43-48.
Jerusalinsky, D., Kornisiuk, E., Alfaro, P., Quillfeldt, J., Alonso, M., Verde, E. R., Cervenansky, C., Harvey, A. (1998) Muscarinic toxin selective for M₄ receptors impairs memory in the rat. NeuroReport. 9, 1407-1411.
Klemm, E., Danos, P., Grumwald, F., Kasper, S., Moller, H. J. and Biersack, H. J. (1996). Temporal lobe dysfunction and correlation of regional cerebral blood flow abnormalities with psychopathology in schizophrenia and major depression—a study with single photon emmission computed tomography. Psychiatry Res.: Neuroimaging, 68 (1), 1-10.
Luby, E. D., Cohen, B. D., Rosenbaum, G., Gottlieb, J. S. and Kelley, R. (1959). Study of a new schizophrenomimetic drug. Sernyl. Arch. Neurol. Psychiatry, 81, 363-369.
Paxinos, G. and Watson, C. (1998). The rat brain in stereotaxic co-ordinates (4^{th} Edition). Academic Press, Sydney.
Potkin, S. G., Buchsbaum, M. S., Jin, Y., Tang, C., Telford, J., Friedman, G., Lottenberg, S., Najafi, A., Gulasekaram, B., Costa, J. & al, e. (1994). Clozapine effects on glucose metabolic rate in striatum and frontal cortex. journal of clinical psychiatry, 55 Suppl B, 63-6.
Schroeder, J., Buchsbaum, M. S., Siegel, B. V., Geider, F. J., Haier, R. J., Lohr, J., Wu, J. & Potkin, S. G. (1994). Patterns of cortical activity in schizophrenia. Psychological Medicine, 24, 947-55.
Schroder, J., Buchsbaum, M. S., Seigel, B. V., Geider, F. J. and Nierthammer, R. (1995). Structural and functional correlates of subsyndromes in chronic schizophrenia. Psychopathology, 28, 38-45.
Seeman, P. (2001) Antipsychotic drugs, dopamine receptors, and schizophrenia. Clinical Neuroscience Research 1, 53-60.
Swerdlow, N. R., Braff, D. L., Taaid, N., Geyer, M. A. (1994) Assessing the validity of an animal model of deficient sensorimotor grating in schizophrenic patients. Arch Gen Psychiatry 51, 139-154.
Vanhoenacker, Guy Haegeman and Josée E. Leysen (2000) 5-HT₇ receptors: current knowledge and future prospects. Trends in Pharmacological Sciences, 21, 70-77
Wolkin, A., Sonfilipo, M., Wolf, A. P., Angrist, B., Brodie, J. D. and Rotrosen, J. (1992). Negative symptoms and hypofrontality in chronic schizophrenia
   Abbreviations

The abbreviations used in this specification are those used within the article Brain in Stereotaxic Coordinates (Paxinos and Watson, 1998).

1, layer I of cortex

2, layers II & III of cortex

3, layers V & VI of cortex

AM, anteromedial thalamus

Au1, primary auditory cortex

V, anteroventral thalamus

Cg (Cg1,-Cg3), anterior cingulate cortex

CM, centromedial thalamic nucleus

DLL, dorsal nucleus of the lateral lemniscus

Ent, entorhinal cortex

Ge, gelatinous nucleus of the thalamus

I, insular cortex

IL, infralimbic cortex

IM, intramedial thalamic nucleus

lO, lateral orbital cortex

M1 & M2, primary and secondary motor cortex

MD, mediodorsal thalamic nucleus

MG, medial geniculates

mO, medial orbital cortex

P, parietal cortex

Pir, piriform cortex

PrL, prelimbic region of the medial prefrontal cortex

PV, paraventriclular thalamic nucleus

Re, nucleus reuniens of the thalamus

Rh, rhomboid nucleus of the thalamus

RSG, retrosplenial cortex

Rt, reticular nucleus of the thalamus (d=dorsal part; v=ventral part)

V2, secondary visual cortex

VCP, ventral cochlear nucleus, posterior

VL, ventrolateral thalamic nucleus

VLL, ventral nucleus of the secondary auditory cortex

VM, ventromedial thalamic nucleus

vO, ventral orbital cortex

1. A pharmaceutical agent having serotonin 5-HT₇ receptor antagonist activity and muscarinic M₄ receptor agonist activity, for use in treating psychotic conditions, wherein the agent does not include compounds having a chemical structure falling within the following definition, namely:
bisarylazepines substituted at the azepine ring portion by a 4-methyl piperazinyl, wherein the aryl moieties are fused to the azepine ring and wherein aryl is phenyl, substituted phenyl, thienyl or substituted thienyl; including optional replacement of an azepine ring carbon atom with a nitrogen atom, or substitution of said ring carbon atom.

2. The pharmaceutical agent according to claim 1 wherein the psychotic condition is schizophrenia and/or bipolar disorder.

3. The pharmaceutical agent according to claim 1 which comprises a mixture of at least two compounds, wherein at least one of said compounds possesses serotonin 5-HT₇ receptor antagonist activity and wherein at least one of said compounds possesses muscarinic M₄ receptor agonist activity.

4. The pharmaceutical agent according to claim 1 which comprises a compound which possesses both serotonin 5-HT₇ receptor antagonist activity and muscarinic M₄ receptor agonist activity.

5. The pharmaceutical agent according to claim 1 which additionally has a low or substantially no dopaminergic D₂ receptor affinity.

6. The pharmaceutical agent according to claim 5 wherein said dopaminergic D₂ receptor affinity is a minimum of at least 5 fold less than the affinity at the muscarinic M₄ and/or serotonin 5-HT₇ receptors.

7. The pharmaceutical agent according to claim 6 wherein said dopaminergic D₂ receptor affinity is at least 50 fold less than the affinity at the muscarinic M₄ and/or serotonin 5-HT₇ receptors.

8. A pharmaceutical agent according to claim 1 for use in therapy.

9. A pharmaceutical formulation comprising a pharmaceutical agent according to claim 1 together with a pharmaceutically acceptable carrier therefor.

10. A method for the preparation of a medicament for the treatment or prophylaxis of schizophrenia and/or bipolar disorder, which comprises mixing the pharmaceutical agent according to claim 1 with a pharmaceutically acceptable carrier.

11. A method of treating psychotic conditions in a patient in need thereof, comprising administering to the patient an effective amount of a pharmaceutical agent according to claim 1.

12. A method of identifying an agent for use in treating psychotic conditions comprising the steps of:
a) providing an agent to be tested;
b) subjecting said agent to one or more test procedures to identify 5-HT₇ receptor antagonist activity and muscarinic M₄ receptor agonist activity of said agent; wherein the desired agent is considered to have been identified when said agent provides a 5-HT₇ receptor antagonist activity and a muscarinic M₄ receptor agonist activity.

13. The method according to claim 12 further comprising the step of subjecting the agent to a test procedure to identify low dopaminergic D₂ receptor affinity.

14. A compound represented by formula (I): 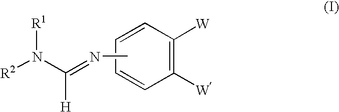
where R¹ and R² independently are a hydrogen atom, a substituted or unsubstituted straight chain or branched chain C₁₋₆ alkyl group or C₁₋₆ alkoxy group, a substituted or unsubstituted C₃₋₈ cycloalkyl group or a C₃₋₈ cycloalkoxy group, or an aralkyl group, or R¹ and R² form, together with the nitrogen atom to which they are bonded, a cyclic amine; W and W′ form, together with the benzene ring to which they are bonded, a fused five-membered, six-membered or seven-membered saturated carbocylic ring being independently unsubstituted, substituted or fully substituted at each carbon atom of the ring by a group —X—R¹³ where X is O, S, SO or SO₂ and R¹³ is a hydrogen atom, a C₁₋₆ alkyl group, an acyl group, or an aroyl group or two of said —X—R¹³ groups, together with the carbon atom in the ring to which they are both bonded, form a C═S group or the following group: 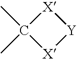
where both of X′ are O or S and Y is a C₁₋₃ alkylene group.

15. The compound according to claim 14, wherein said cyclic amine is substituted by a halogen atom, a C₁₋₆ alkyl group or a C₁₋₆ alkoxy group.

16. The compound according to claim 14 wherein said cyclic amine is fused with a benzene ring.

17. The compound according to claim 16 wherein said benzene ring is substituted by one or two halogen atoms, C₁₋₆ alkyl groups or C₁₋₆ alkoxy groups.

18. The compound according to claim 14 represented by the following formulae (II), (III) or (IV): 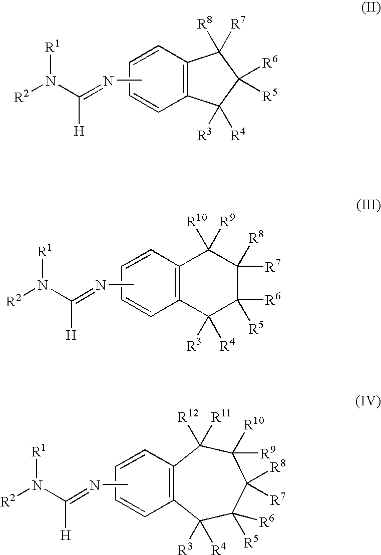
wherein R¹ and R² independently are a hydrogen atom, a substituted or unsubstituted straight chain or branched chain C₁₋₆ alkyl group or C₁₋₆ alkoxy group, a substituted or unsubstituted C₁₋₆ cycloalkyl group or a C₁₋₆ cycloalkoxy group, or an aralkyl group, or R¹ and R² form, together with the nitrogen atom to which they are bonded, a cyclic amine; R³, R⁴, R⁵, R⁶, R⁷, R⁸, R⁹, R¹⁰, R¹¹, and R¹² are independently a hydrogen atom or the group —X—R¹³ wherein X is O, S, SO or SO₂ and R¹³ is a hydrogen atom, a C₁₋₆ alkyl group, an acyl group, or an aroyl group.

19. The compound according to claim 18 wherein R³ and R⁴, R⁵ and R⁶, R⁷ and R⁸, R⁹ and R¹⁰, and/or R¹¹ and R¹² together with the carbon atom in the ring to which they are both bonded, form a C═S group or the following group: 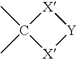
wherein both of X′ are O or S and Y is a C₁₋₃ alkylene group.

20. The compound according to claim 18 wherein R¹ and R² form together with the nitrogen atom to which they are bonded, a four-membered, five-membered or six-membered cyclic amine.

21. A compound according to claim 20 wherein said six-membered cyclic amine is fused with a benzene ring.

22. The compound according to claim 18 wherein R¹ and R² are a C₁₋₆ alkyl group.

23. The compound according to claim 14 which possesses serotonin 5-HT₇ receptor antagonist activity and/or muscarinic M₄ receptor agonist activity.

24. The compound according to claim 23 which additionally has a low or substantially no dopaminergic D₂ receptor affinity.

25. The compound according to claim 14 for use in therapy.

26. A pharmaceutical formulation comprising a compound according to claim 14 admixed with a pharmaceutically acceptable carrier.

27. A method for the preparation of a medicament for the treatment or prophylaxis of schizophrenia and/or bipolar disorder, which comprises mixing the compound according to claim 14 with a pharmaceutically acceptable carrier.

28. A method of treating psychotic conditions in a patient in need thereof, comprising administering to the patient an effective amount of a compound according to claim 14.

29. The pharmaceutical agent according to claim 3 wherein the psychotic condition is schizophrenia and/or bipolar disorder.

30. The pharmaceutical agent according to claim 4 wherein the psychotic condition is schizophrenia and/or bipolar disorder.

31. The pharmaceutical agent according to claim 5 wherein the psychotic condition is schizophrenia and/or bipolar disorder.

32. The pharmaceutical agent according to claim 6 wherein the psychotic condition is schizophrenia and/or bipolar disorder.

33. The pharmaceutical agent according to claim 7 wherein the psychotic condition is schizophrenia and/or bipolar disorder.

34. The pharmaceutical agent according to claim 8 for use in therapy for schizophrenia and/or bipolar disorder.

35. The pharmaceutical formulation according to claim 9 for use in therapy for schizophrenia and/or bipolar disorder.

36. The method according to claim 11 wherein the psychotic condition is schizophrenia and/or bipolar disorder.

37. The compound according to claim 19 wherein R¹ and R² form together with the nitrogen atom to which they are bonded, a four-membered, five-membered or six-membered cyclic amine.

38. The compound according to claim 37 wherein said six-membered cyclic amine is fused with a benzene ring.

39. The method according to claim 28 wherein the psychotic condition is schizophrenia and/or bipolar disorder.
